(19) United States
(12) Patent Application Publication
(10) Pub. No.: US 2022/0157456 A1
(43) Pub. Date: May 19, 2022

(54) INTEGRATED HEALTHCARE PLATFORM

(71) Applicant: SolaVieve Technologies GmbH, Heidelberg (DE)

(72) Inventors: Joshua LUCKOW, Heidelberg (DE); Alexander RISSLAND, Heidelberg (DE); Juliana ASCOLANI, Heidelberg (DE)

(21) Appl. No.: 17/526,519

(22) Filed: Nov. 15, 2021

Related U.S. Application Data
(60) Provisional application No. 63/278,910, filed on Nov. 12, 2021.
(60) Provisional application No. 63/113,364, filed on Nov. 13, 2020.

(51) Int. Cl. G16H 40/67; G16H 80/00; G16H 10/20; G16H 10/60; G06N 5/02; G06N 5/04

(52) U.S. Cl. CPC G16H 40/67; G16H 80/00; G06N 5/04; G16H 10/60; G06N 5/022; G16H 10/20

(57) ABSTRACT

A digital health platform is configured to provide users with access to health and wellness education and data and to facilitate user interaction with health and wellness practitioners and/or virtual coaching. The platform may include an artificially intelligent virtual AI coach configured to provide suitable statements and/or recommendations to users in response to user input (e.g., user responses to questions or prompts). In some examples, the platform includes analytics configured to derive insights based on user data and/or any other suitable data. In some examples, the platform provides information and services related to mental, physical, spiritual, social, environmental, and economic dimensions of wellness.

### CROSS-REFERENCES

The following applications and materials are incorporated herein, in their entireties, for all purposes: U.S. Provisional Patent Application Ser. No. 63/113,364, filed Nov. 13, 2020, and U.S. Provisional Patent Application Ser. No. 63/278,910, filed Nov. 12, 2021.

### FIELD

This disclosure relates to systems and methods for providing healthcare and wellness services, products, and information digitally.

### INTRODUCTION

Existing digital healthcare solutions are highly fragmented. For example, known digital healthcare systems typically address isolated aspects of health such as mental health or medical treatment, and fail to integrate the various facets of an individual person's health, lifestyle, and wellbeing, e.g., mental, physical, spiritual, social, environmental, economic. Furthermore, known digital health care systems are personalized to only a small extent, limiting their effectiveness. These known systems are most often focused on disease treatment and management. These known systems are most often not focused on informing, guiding, and empowering individuals to take personal ownership of their health and wellbeing and focus on prevention through healthy lifestyle practices.

Another drawback of known healthcare systems is that seeking preventive and/or holistic health care from health practitioners tends to be costly due to, e.g., lack of insurance reimbursement, and demand can greatly surpass supply. Physicians and other high skilled practitioners are often overbooked, difficult to get unscheduled appointments with, quite expensive, and provide only small amounts of consultation time, for example 5-7 minutes. The success of these sessions is often measured by volume (e.g., how many appointments a practitioner can see in one day), rather than disease prevention. In many cases, health insurance does not cover certain fields, especially in the area of preventive healthcare. However, there is an increasing demand for preventive healthcare services and self-help which needs to be met in a cost-effective manner. Additionally, preventive healthcare services that rely only on practitioner-to-patient interactions are extremely cost- and resource-intensive, as practitioners' time is limited and must be dedicated to one user at a time.

Better solutions are needed for improving availability of practitioner sessions and providing individuals with personalized, holistic care.

### SUMMARY

The present disclosure provides systems, apparatuses, and methods relating to digital health care and wellness services.

In some examples, a computer-implemented health platform may include a server including a server-side program configured to execute a virtual coach including an AI system; a first client device including a client-side program in communication with the server-side program via a computer network; wherein the client-side program and the server-side program are configured to facilitate a chat session between a user of the first client device and the virtual coach, and wherein facilitating the chat session includes: receiving, via a user interface executed at the first client device by the client-side program, a first user message input by the user; using the virtual coach, determining, based on first data including the first user message and user-specific data stored at a memory store in communication with the server, a first virtual coach message to be presented to the user; presenting, via the user interface, the first virtual coach message; receiving, via the user interface, a second user message input by the user; using the virtual coach, determining, based at least on the second user message, a second virtual coach message to be presented to the user; and presenting, via the user interface, the second virtual coach message.

In some examples, a computer-implemented method for providing digital health and wellness services may include storing, at a memory store, user data relating to a wellness behavior of a user; receiving, at a processor in communication with the memory store, a first chat message input by the user at a user computing device; automatically deriving, based on the stored user data and the first chat message, using an artificial intelligence (AI) coach executed by the processor, a recommended action for the user to take to improve their wellness; and presenting, at the user computing device, a second chat message including the recommended action.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is a schematic diagram depicting an illustrative digital health platform in accordance with aspects of the present disclosure.

FIG. 2 is a schematic diagram depicting an illustrative data flow of an illustrative digital health platform, in accordance with aspects of the present teachings.

FIG. 3 is a schematic diagram depicting an illustrative input mask of the data flow of FIG. 2.

FIG. 4 is a schematic diagram depicting an illustrative chat module of the data flow of FIG. 2.

FIG. 5 is a schematic diagram depicting an illustrative deployment module of the data flow of FIG. 2.

FIG. 6 is a schematic diagram depicting aspects of an illustrative wellness assessment of a digital health platform, in accordance with aspects of the present teachings.

FIG. 7 is a diagram depicting aspects of an illustrative report based on a user's responses to the wellness assessment of FIG. 6.

FIG. 8 is a schematic diagram depicting aspects of an illustrative health literacy assessment of a digital health platform, in accordance with aspects of the present teachings.

FIG. 9 is a diagram depicting an illustrative screenshot of a user application of a digital health platform, in accordance with aspects of the present teachings.

FIG. 10 is a diagram depicting another illustrative screenshot of a user application of a digital health platform, in accordance with aspects of the present teachings.

FIG. 11 is a flowchart depicting steps of an illustrative method for providing digital health and wellness services according to the present teachings.

FIG. 12 is a schematic diagram depicting an illustrative data processing system, in accordance with aspects of the present teachings.

FIG. 13 is a schematic diagram depicting an illustrative network data processing system, in accordance with aspects of the present teachings.

FIG. 14 is a schematic diagram depicting an illustrative system for machine learning training and operation.

### DETAILED DESCRIPTION

Various aspects and examples of a digital healthcare platform are described below and illustrated in the associated drawings. Unless otherwise specified, a digital healthcare platform in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

This Detailed Description includes the following sections, which follow immediately below: (1) Definitions; (2) Overview; (3) Examples, Components, and Alternatives; (4) Advantages, Features, and Benefits; and (5) Conclusion. The Examples, Components, and Alternatives section is further divided into subsections, each of which is labeled accordingly.

### Definitions

The following definitions apply herein, unless otherwise indicated.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

“AKA” means “also known as,” and may be used to indicate an alternative or corresponding term for a given element or elements.

“Processing logic” describes any suitable device(s) or hardware configured to process data by performing one or more logical and/or arithmetic operations (e.g., executing coded instructions). For example, processing logic may include one or more processors (e.g., central processing units (CPUs) and/or graphics processing units (GPUs)), microprocessors, clusters of processing cores, FPGAs (field-programmable gate arrays), artificial intelligence (AI) accelerators, digital signal processors (DSPs), and/or any other suitable combination of logic hardware.

“Providing,” in the context of a method, may include receiving, obtaining, purchasing, manufacturing, generating, processing, preprocessing, and/or the like, such that the object or material provided is in a state and configuration for other steps to be carried out.

In this disclosure, one or more publications, patents, and/or patent applications may be incorporated by reference. However, such material is only incorporated to the extent that no conflict exists between the incorporated material and the statements and drawings set forth herein. In the event of any such conflict, including any conflict in terminology, the present disclosure is controlling.

Overview In general, a digital healthcare platform in accordance with aspects of the present teachings may be configured to provide a plurality of health-related services to users. The platform centralizes and integrates a variety of health-related data in an integrated and dynamically adjustable database. The database may include user-specific data determined directly by user input (e.g., in response to assessment questions) and/or user-specific data determined indirectly by analyzing user behavior and/or user-input data (e.g., insights derived by machine-learning algorithms). In some examples, the database further includes aggregated directly or indirectly obtained user data and practitioner data for all platform users, or for subsets of platform users. The database may further include general health information (e.g., recommendations for nutrition, sleep hygiene, medical screenings, vaccinations, and so on); data received from user devices (e.g., smartphones, wearable devices such as smartwatches, etc.); data relating to user behavior (e.g., metadata about their interactions with the platform, settings of user devices, social media use, etc.); and/or any other suitable data. Examples of data suitable for use in the database and/or other aspects of the platform are further described below.

In some examples, the platform includes a chat module configured to facilitate interaction between users and health practitioners, and between users and an artificial-intelligence (AI) virtual coach. The health practitioners and AI virtual coach may use the information in the database to formulate a recommendation for a user. Practitioners associated with the platform may include traditional practitioners of Western medicine (e.g., physicians, physicians' assistants, nurses, psychologists and/or psychiatrists, dentists, etc.), complementary medicine (e.g., nutritionists, physiologists, physical therapists, chiropractors, athletic trainers, personal trainers, naturopathic physicians, etc.), as well as, Eastern medicine practitioners (e.g., Ayurvedic physicians and Traditional Chinese Medicine practitioners, etc.), life coaches, executive coaches, spiritual healers, creative artists, environmental health specialists, financial health advisors, and/or any other practitioners suitable for providing health- and wellness-related services to users via the platform. In some examples, the platform includes a large library of articles, videos, podcasts, and other forms of materials available to clients and/or to health practitioners.

In some examples, the platform provides health tracking data on, e.g., health status and health literacy, in multiple health determinants to provide a whole-person health assessment: mental, physical, spiritual, social, environmental, economic. The platform may, for example, provide data tracking activities, services, and products that the users have done and/or used to show them the progress they have made.

In some examples, the platform includes a progressive web application (PWA) configured to facilitate user interaction with the platform. The PWA may use web-related technologies (e.g., HTML, CSS, JavaScript, and/or the like). The PWA may combine both web and native applications' features, enabling distribution on mobile and desktop devices, as well as wearables such as smartwatches and bands. Alternatively, or additionally, at least some functions of the platform may be accessible via standalone software applications, internet browsers, mobile apps, and/or the like.

In some examples, the platform further includes social media features, integration with dedicated or third-party technology (e.g., pedometers, heart-rate monitors, smart watches, and/or any other suitable devices), and/or any other suitable integration feature(s).

Aspects of the digital health platform may be embodied as a computer method, computer system, or computer program product. Accordingly, aspects of the platform may take the form of an entirely hardware embodiment, an entirely software embodiment (including firmware, resident software, micro-code, and the like), or an embodiment combining software and hardware aspects, all of which may generally be referred to herein as a “circuit,” “module,” or “system.” Furthermore, aspects of the platform may take the form of a computer program product embodied in a computer-readable medium (or media) having computer-readable program code/instructions embodied thereon.

Any combination of computer-readable media may be utilized. Computer-readable media can be a computer-readable signal medium and/or a computer-readable storage medium. A computer-readable storage medium may include an electronic, magnetic, optical, electromagnetic, infrared, and/or semiconductor system, apparatus, or device, or any suitable combination of these. More specific examples of a computer-readable storage medium may include the following: an electrical connection having one or more wires, a portable computer diskette, a hard disk, a random-access memory (RAM), a read-only memory (ROM), an erasable programmable read-only memory (EPROM or Flash memory), an optical fiber, a portable compact disc read-only memory (CD-ROM), an optical storage device, a magnetic storage device, and/or any suitable combination of these and/or the like. In the context of this disclosure, a computer-readable storage medium may include any suitable non-transitory, tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, and/or any suitable combination thereof. A computer-readable signal medium may include any computer-readable medium that is not a computer-readable storage medium and that is capable of communicating, propagating, or transporting a program for use by or in connection with an instruction execution system, apparatus, or device.

Program code embodied on a computer-readable medium may be transmitted using any appropriate medium, including but not limited to wireless, wireline, optical fiber cable, RF, and/or the like, and/or any suitable combination of these.

Computer program code for carrying out operations for aspects of the digital health platform may be written in one or any combination of programming languages, including an object-oriented programming language (such as Java, JavaScript, C++, Angular), conventional procedural programming languages (such as C), and functional programming languages (such as Haskell). Mobile apps may be developed using any suitable language, including those previously mentioned, as well as Objective-C, Swift, C#, HTML5, and the like. The program code may execute entirely on a user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), and/or the connection may be made to an external computer (for example, through the Internet using an Internet Service Provider).

Aspects of the platform may be described below with reference to flowchart illustrations and/or block diagrams of methods, apparatuses, systems, and/or computer program products. Each block and/or combination of blocks in a flowchart and/or block diagram may be implemented by computer program instructions. The computer program instructions may be programmed into or otherwise provided to processing logic (e.g., a processor of a general purpose computer, special purpose computer, field programmable gate array (FPGA), or other programmable data processing apparatus) to produce a machine, such that the (e.g., machine-readable) instructions, which execute via the processing logic, create means for implementing the functions/acts specified in the flowchart and/or block diagram block(s).

Additionally or alternatively, these computer program instructions may be stored in a computer-readable medium that can direct processing logic and/or any other suitable device to function in a particular manner, such that the instructions stored in the computer-readable medium produce an article of manufacture including instructions which implement the function/act specified in the flowchart and/or block diagram block(s).

The computer program instructions can also be loaded onto processing logic and/or any other suitable device to cause a series of operational steps to be performed on the device to produce a computer-implemented process such that the executed instructions provide processes for implementing the functions/acts specified in the flowchart and/or block diagram block(s).

Any flowchart and/or block diagram in the drawings is intended to illustrate the architecture, functionality, and/or operation of possible implementations of systems, methods, and computer program products according to aspects of the platform. In this regard, each block may represent a module, segment, or portion of code, which comprises one or more executable instructions for implementing the specified logical function(s). In some implementations, the functions noted in the block may occur out of the order noted in the drawings. For example, two blocks shown in succession may, in fact, be executed substantially concurrently, or the blocks may sometimes be executed in the reverse order, depending upon the functionality involved. Each block and/or combination of blocks may be implemented by special purpose hardware-based systems (or combinations of special purpose hardware and computer instructions) that perform the specified functions or acts.

### EXAMPLES, COMPONENTS, AND ALTERNATIVES

The following sections describe selected aspects of illustrative digital health platforms as well as related systems and/or methods. The examples in these sections are intended for illustration and should not be interpreted as limiting the scope of the present disclosure. Each section may include one or more distinct embodiments or examples, and/or contextual or related information, function, and/or structure.

### A. Illustrative Digital Platform

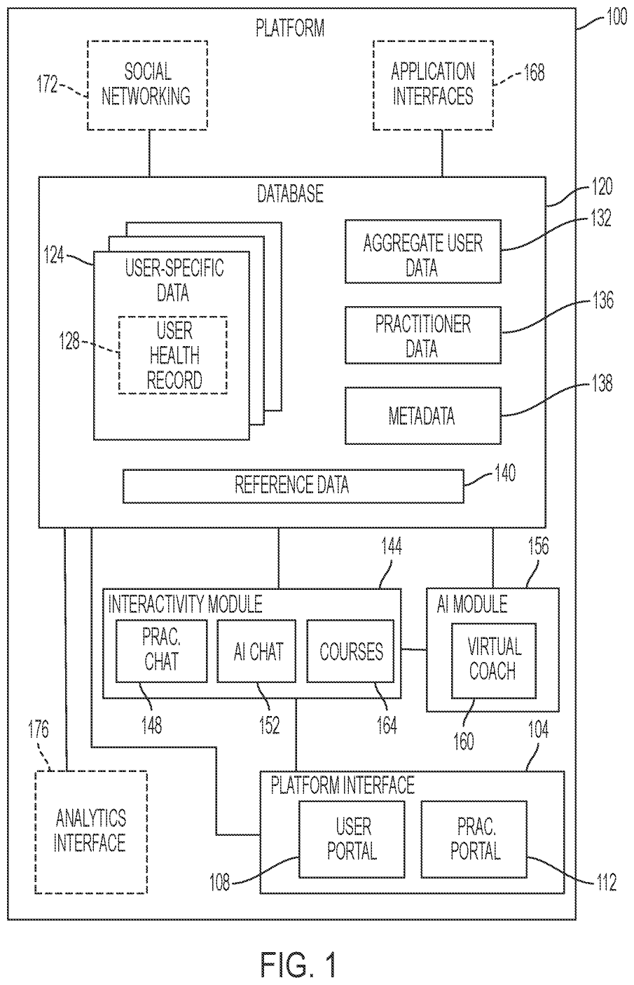

With reference to FIG. 1, this section describes an illustrative digital platform 100, which is an example of the platform described above.

Platform 100 includes a platform interface 104 configured to facilitate user access to various aspects of the platform. For example, platform interface 104 may allow a user to log in to the platform, chat with a provider or AI coach, browse medical and wellness information, take health status and knowledge-level assessments, and/or access any other suitable feature(s) of the platform. Platform interface 104 may include a user portal 108 facilitating access for end users and a practitioner portal 112 facilitating access for doctors, nurses, therapists, coaches, and/or other experts providing services and/or information to users. Portals 108 and 112 may be configured to allow users or practitioners respectively to log in to the portal (e.g., using unique credentials) and access appropriate data and/or features. In some examples, each user (or groups of users) accesses the platform using a respective smartphone, computer, and/or other suitable data-processing system connected by a network (e.g., the Internet, a local area network, and/or any other suitable network) to one or more servers or other suitable system(s) hosting aspects of platform 100.

Platform 100 further includes a database 120 including data stored on one or more data storage devices. Database 120 includes user-specific data 124 including data associated with specific users of the platform. For example, user-specific data may be stored in association with respective user IDs in database 120. User-specific data 124 for each user may include, e.g., user profile/demographics (name, age, body measurements, nationality, etc.); vital signs (e.g., measured by a user and/or automatically communicated to the database by a measuring device in communication with the platform); diagnostic results; health history; family health history; lifestyle history (e.g., exercise habits, smoking, drug use, and/or any other suitable information about the user's past or present lifestyle); recordings, notes, and/or summaries from consultation sessions with a virtual coach or human practitioner; transcripts of conversation the user and practitioners or other users of the platform; answers provided by the user in response to questions posed by a practitioner, AI virtual coach, or by a survey or assessment presented to the user by the platform; questions submitted by the user (e.g., questions submitted to a practitioner, to the virtual coach, search queries in the database, etc.); the user's present or historical mood (determined by direct user input, by analytics and/or machine-learning based on information provided by the user and/or the user's interaction with the platform, and/or determined in any other suitable manner); the user's interests, affirmations, intentions, and goals (determined, e.g., directly based on user-identified interests and/or indirectly based on other user input or behavior); general aspects of a user's life including, e.g., passions, undertakings, responsibilities, social interactions, limitations and constraints (physical, mental, financial, time, etc.); and metadata relating to the user's use of the platform (e.g., their GPS location when participating in sessions with the virtual coach and/or human practitioners (e.g., whether they are at work, at home, traveling, out shopping, etc.), the length of time taken by a user to provide responses to questions (e.g., from the virtual coach or a practitioner, from a prompt or assessment by the platform), the frequency with which the user accesses the platform or aspects of the platform (e.g., an hourly, daily, weekly, monthly, and/or yearly rate of usage), times of day, week, month, or year when the user accesses the platform, and/or any other suitable data.

User-specific data 124 optionally includes a user health record 128 associated with the user. User health record 128 may include any or all of the data described above. In some examples, aggregated and analyzed benchmarking data from the database is also included. Data may be added to user health record 128 by the user, by health practitioners, by administrative staff, and/or by the virtual coach and/or other AI modules. Internal data control structures may be configured to monitor the suitability of at least certain modifications made to the record by AI (e.g., suggested medications). In some examples, data of user health record 128 is obtained from social networking aspects of the platform, from electronic devices (e.g., wearable devices), from third-party websites or applications via application interfaces, and/or any other suitable source.

In some examples, user health record 128 includes information in addition to the types of information typically included in traditional medical records or medical charts. For example, user health record 128 may include information related to mental, physical, spiritual, social, environmental, and economic aspects of a user's health and/or wellbeing.

Database 120 further includes aggregate user data 132. Aggregate user data 132 includes aggregated (optionally, anonymized) data corresponding to the user-specific data of all platform users. Results of analytics performed on the aggregated data may be stored in aggregate user data 132. This facilitates identification of statistical information about the health of users of the platform and/or the manner in which users use the platform. For example, analytics performed on aggregate user data 132 may identify trends in the health (e.g., sleep habits, exercise habits, self-reported illness) of users, treatments recommended by practitioners or the virtual coach that result in improvement to user wellbeing, and/or any other suitable insights.

Database 120 further includes practitioner data 136 associated with practitioners who provide services to users via the platform. Practitioner data 136 may include demographic information about practitioners; practitioner profiles, skills, licensure, certification, and/or insurance coverage(s); services offered by the practitioner; records of services actually provided by the practitioner; user ratings of and feedback on practitioner(s); progress notes entered by practitioners; collaborations between health professionals within the same discipline (e.g., physician to physician, nutritionist to nutritionist, etc.) and across disciplines (e.g., physician to nutritionist, psychologist to physiologist, etc.), individual pricing or practitioners, availability of practitioners, and/or any other suitable data relating to individual and/or aggregated practitioners.

Database 120 further includes metadata 138. Metadata 138 includes data tracked by platform 100 relating to user sessions (e.g., how long users interact with the platform, the GPS location of users during their interaction with the platform, which features users most frequently interact with, etc.); to practitioner sessions (e.g., how long practitioners interact with the platform, the GPS location of practitioners at the time of interacting with the platform, the frequency with which they interact with the platform, which data in database 120 they access, etc.); and/or other suitable metadata. In some examples, metadata is also stored as part of user-specific data 124 and/or practitioner data 136, as appropriate.

Database 120 further includes reference data 140. Reference data 140 can include any suitable data of interest to users and/or practitioners and related to health, wellness, and/or general lifestyle improvement. Reference data 140 may be accessed by users, practitioners, and/or AI aspects of the platform to increase understanding and/or recommended practices for any suitable wellness issues. Reference data 140 may include, e.g., health knowledgebases (including, e.g., the following areas of health & wellness: Western medicine; complementary and alternative medicine (CAM) and Eastern medicine; Ayurvedic medicine; Traditional Chinese Medicine; homeopathy; naturopathy; chiropractic knowledge; behavioral health and mental health; exercise science and personal training; nutritional science; spiritual, religious-oriented, and creativity-oriented practices; body healing and energy healing; environmental health, conservation, conscious consumption, etc.; financial health, purposeful work, work-life balance, etc.; therapeutic methodologies of behavioral change (e.g., Solution Focused Brief Therapy (SFBT)); and/or organization management, e.g. time and resource management.

These and/or any other suitable knowledgebases may include health facts and statistics; risk factors of disease; health knowledge, research studies and/or outcomes; best practices; prevention and lifestyle practices; questions suitable for a practitioner to ask a patient in a consultation, or vice versa; symptom triage questioning; diagnostic algorithms; user response options; curricula for health education courses; statements available for the virtual coach and/or other interactive features of the platform to present to a user (e.g., facts, figures, general statements, questions, and so on); example and/or historical user responses to these or other statements; infographics, videos, articles, and/or other media; scientific research; facts and statistics (e.g., corresponding to one or more regions and/or nations, and/or worldwide); metadata on sources of information (publishers, authors, publication dates, etc.); assessments of the trustworthiness of research (based, e.g., on amounts and/or qualities of evidentiary support for the research, a reputation of the researcher(s), and/or any other indicator(s) of reliability); categorization of each piece of research (e.g., as relating to nutrition, mental health, and/or another aspect of wellbeing; as relating to a particular demographic; as relating to a particular modality of research; and/or any other suitable categorizations); connections, overlaps, and/or adjacencies that an article or other piece of information from a first field has to one or more other fields (e.g., a report on nutritional research that suggests implications for behavioral health research); and/or any other suitable information, including source information related to obtained and analyzed research, which may be used to, e.g., ensure the integrity, accuracy, completeness, and reliability of the research.

Platform 100 includes an interactivity module 144 configured to allow users to interact with practitioners and/or with AI features of the platform, such as the virtual coach. As an example, interactivity module 144 may have a concierge servicing function configured to allow a user to schedule an appointment with a practitioner.

As another example, interactivity module 144 may be configured to provide daily check-ins to a user, wherein platform 100 presents one or more questions, reminders, and/or affirmations to a user, and the user optionally responds with information and/or confirmation. As one example of a daily check-in, platform 100 may display a prompt to a user (via a user interface of a smartphone or other data-processing system with which the user is accessing the platform) asking the user to assess their mood. The user can respond by selecting a rating, inputting a text description, and/or by providing any other suitable response. The user's response (and optionally, related metadata such as time elapsed between the appearance of the prompt and receipt of the user's response, the GPS location of the user at the time the response is received, etc.) are stored in user-specific data 124.

Interactivity module 144 includes a practitioner chat module 148 configured to facilitate conversation between one or more users and one or more practitioners. Practitioner chat module 148 may comprise any suitable software configured to facilitate text-based real-time chat, video chat, voice-only chat, and/or any other suitable type of conversation. In some examples, interactions between users and practitioners are facilitated by communications technology configured to be compliant with HIPPA and/or any other suitable laws, regulations, or best practices. In some examples, third-party communication software is embedded in platform 100 using an application programming interface (API) integration. One example of suitable technology is the communication software sold under the name Sendbird. In examples using integrated third-party software, stored data related to conversations between users and practitioners is stored in database 120.

Interactivity module 144 further includes an AI chat module 152 configured to facilitate conversation between one or more users and one or more artificially intelligent features of the platform. AI chat module 152 may comprise any suitable software configured to facilitate text-based real-time chat, video chat, voice-only chat, and/or any other suitable type of conversation between a user and an AI or other automated feature of the system. In some examples, AI chat module 152 is configured to facilitate the presentation of infographics, videos, and/or audio recordings to the user (e.g., as part of an interaction between the user and an AI feature of the system).

An AI module 156 of platform 100 is configured to provide appropriate statements, questions, responses, and/or other suitable communications to AI chat module 152. Suitable communications may be identified by AI module 156 based on user input to AI chat module 152, user-specific data 124, and/or any other suitable data using machine learning, artificial intelligence, rule-based decision-making, and/or any other suitable algorithms and/or methods.

In some examples, AI module 156 (and/or any other suitable components of platform 100) is configured to perform big data analytics on data stored in database 120. For example, the module may be configured to use machine learning to identify patterns, trends, associations, and insights in the data, and/or to provide conclusions and solutions to the users and practitioners based on the identified insights. The conclusions and solutions for the practitioners may enhance their service capabilities with the users, while also increasing their personal knowledgebase.

As another example, analytics performed by platform 100 may identify correlations and/or commonalities among various health disciplines, breaking down traditional siloed barriers that exist between the disciplines.

As another example, platform 100 may be configured to use predictive analytics and/or behavioral analytics to prevent disease and help ensure users are living well. Such analyses may also be used to train and further develop the skills and knowledge of practitioners serving users via the platform.

Analytics performed by platform 100 may also be used to identify promising and/or interesting areas for further research and study.

AI module 156 includes a virtual coach 160 comprising an AI system configured to interact with users (e.g., via AI chat module 152 and/or any other suitable communication method). From a user's point of view, virtual coach 160 acts as a coach and/or advisor who is available 24/7 and who can listen to and understand the user's input, and offer solutions that are practical, holistic, and timely. Analytics performed by platform 100 may be further used to train the virtual coach, as well as be offered to the public and public institutions as outlined below.

Virtual coach 160 may be implemented using any suitable technology, including machine learning, natural language processing, deep learning, and/or any other suitable technology. For example, virtual coach 160 may be configured to use machine-learning algorithms to determine a quality of the user (e.g., their intent, mood, and/or any other suitable feature and/or property) based on data input by the user (e.g., the user's side of the chat conversation) and/or metadata associated with the data input by the user, and to determine a suitable response to the user based on the determined quality. Machine-learning algorithms trained on data of database 120 may allow virtual coach 160 to establish effective ongoing coaching relationship with users, provide users with meaningful and scientifically based health content, assess user health and identify behavioral patterns and insights, and guide and assist users toward living healthier lives and taking ownership of their health.

In some examples, virtual coach 160 is implemented using virtual assistant and/or chatbot technology, including conversational interactive voice response (IVR). This technology may allow the virtual coach to understand user expressions, match them to intents, and extract structured data. An example of a suitable technology is the Dialogflow system provided by Google. A third-party chatbot technology may be integrated with a PWA or native application of platform 100 using one or more APIs.

Machine-learning aspects of virtual coach 160 (and/or any other portions of AI module 156) may be trained using any suitable data. Based on training data, virtual coach 160 develops a model for predicting user intent based on expressions input by the user into the chat. The virtual coach is configured to provide a suitable response based on predicted user intent. Suitable responses are determined by virtual coach 160 based on, e.g., knowledgebases from a variety of health disciplines (e.g., western medicine, eastern medicine, nutrition, psychology, physiology, theology and religious studies, complementary medicine, homeopathy, etc.), therapeutic methodologies of behavioral change, time management, organizational management, concierge servicing, and/or any other suitable methods and/or principles. Suitable responses may further include directing users to live practitioners and/or to products available for purchase. Virtual coach 160 may be configured to identify, based on user input, one or more conversational tones, vocabulary, syntax complexity, and/or other suitable aspects of communication that a user will find approachable, reassuring, understandable, and/or trustworthy.

In some examples, virtual coach 160 is configured to conduct a daily check-in with a user, as described above. The daily check-in allows the virtual coach to assess a user's current health status, mood, daily issues, achievement of goals, setting of affirmations/goals, etc. Check-in sessions can be general in nature and/or cover one or more specific aspects of health (e.g., one of the following set of six health dimensions: mental, physical, spiritual, social, environmental, and economic wellness). The virtual coach is configured to access and use any suitable user-specific data to guide the daily check-in sessions (e.g., sleeping patterns input by a user during a previous course on sleep; eating patterns describe by a user during practitioner session, etc.). The virtual coach may additionally or alternatively be configured to access and use other types of data to guide the sessions, such as patterns, trends, associations, and insights derived from any suitable data within database 120. In some examples, based on data stored within database 120, the virtual coach speaks about trends in the user base and/or general population, such as fear generated in response to COVID-19 and/or any other suitable current event. In some examples, the virtual coach determines suitable responses to a user during a daily check-in in a rule-based manner and transitions to a self-learning method of decision-making based on user input (e.g., text and/or voice commands), as well as secondary sources (third party or proprietary), such as wearable devices.

In some examples, virtual coach 160 is configured to provide guide users through one or more courses 164. Each course 164 may comprise an interactive session dedicated to one or more specific topics (e.g., sleep habits, stress management, etc.). Platform 100 may include a plurality of courses 164 covering a wide array of health topics. A course 164 may include health facts and/or statistics, risk factors of disease, health knowledge, research studies/outcomes, best practices, prevention and lifestyle practices, practitioner-to-client consultation questions, symptom triage questioning, and/or diagnosis algorithms. During a course, the virtual coach may provide a user with a menu of responses to select from. In some examples, the courses are implemented using virtual assistant and/or chatbot technology.

In some examples, the virtual coach determines suitable responses to a user during a course in a rule-based manner and transitions to a self-learning method of decision-making based on user input (e.g., text and/or voice commands). The courses are facilitated by creating a dialogue between the user and the virtual coach. This technique enables courses to be personalized to a user based on the user's responses. In turn, the user responses become part of a dataset used to train the virtual coach, effectively increasing the knowledgebase of the virtual coach (and/or other AI aspects of the platform).

During a course 164, virtual coach 160 may present a user with a series of questions and/or course topics, and may provide the user with information and/or advice based on user response. Interaction between the user and the virtual coach during a course session (and/or during use of AI chat 152) may be rule-based and/or machine-learning-based, and may transition between rule-based and machine-learning as needed (e.g., the virtual coach may use rule-based decision-making if it is unable to determine a suitable response with sufficient confidence using machine learning).

When conducting a virtual coaching session with a user (e.g., via AI chat 152), the virtual coach accesses any suitable data of database 120. For example, the virtual coach may access data associated with a user's use of courses 164 and/or daily check-ins; the user's health status, responses to any other questions input via the platform; user-selected affirmations; user-selected goals (in progress or achieved); data associated with practitioner sessions; data associated with wearable devices, third-party interfaces, social media sessions; metadata; and/or any other suitable data.

User responses to virtual coach 160 are stored in database 120. Data associated with actions taken by a user following an interaction with virtual coach 160 (e.g., booking a service or a course, purchasing a product, reading a recommended article, etc.) is stored in database 120. Metadata associated with user interactions with virtual coach 160 (e.g., time of day of coaching sessions and/or courses, duration, geographic location, etc.) is stored in database 120.

In some examples, platform 100 is configured to interface with and obtain data from proprietary and/or third-party devices such as wearable devices (e.g., pedometers, smartwatches, smart clothing, etc.), fitness trackers, sleep trackers, biofeedback devices, and/or any other suitable devices from which wellness-related data may be obtained. Similarly, platform 100 may be configured to receive data representing diagnostic results obtained by diagnostic equipment (e.g., cardio metabolic testing equipment, resting metabolic rate equipment, sleep study equipment, done density/body composition equipment, etc.). The data obtained from these and/or any other suitable devices is stored in database 120 and may be accessible by any suitable aspect of platform 100. Obtained data may include, e.g., quality and quantity of sleep, fitness achievements, biofeedback readings on stress, metabolic functioning, etc. The obtained data may be used by the virtual coach, practitioners, and/or users to obtain an understanding of the user's health, lifestyle, and progress indicators. In some examples, the obtained data is aggregated, and the aggregate data may provide an understanding on usage patterns of platform 100 and/or the devices, and/or population-level findings on health status, lifestyle, and progress indicators.

In some examples, platform 100 may be configured to permit diagnostic equipment, wearable devices, and/or any other suitable proprietary and/or third-party devices to access suitable portions of database 120. Data to which devices are permitted access may include, e.g., a lifestyle patterns and health status, demographic data relating to the user, and/or any other suitable data. Platform 100 may be configured to obtain consent from a user before permitting a device to access the user's data.

In some examples, platform 100 includes application interfaces 168 configured to interface with other software applications (e.g., third-party applications and/or proprietary applications) used by users and/or practitioners. For example, the platform may be configured to connect with users' digital calendars and observe schedules that may be too busy, workloads that are unsustainable, or lack of balance with family time, lack of restoration time, etc. As another example, the platform may be configured to connect with users' third-party social media accounts (with the users' explicit permission) and help them observe communication patterns and usage patterns. This helps users to see their own behaviors and provide opportunities for change and improvement.

In some examples, applications communicating with platform 100 via interfaces 168 are allowed to access database 120 to obtain demographic data, lifestyle patterns, health status, and/or any other suitable data. User consent may be required for the applications to access the database. In some examples, data is mined from the applications and stored in database 120.

In some examples, platform 100 includes one or more social networking features 172. Social networking 172 may be configured to allow users to communicate with each other (e.g., via chats, message boards, and/or other suitable methods), to create profiles, to share their health status, accomplishments, goals, and/or any other suitable data with other users, and/or to perform any other suitable social networking functions. This may increase user engagement with platform 100 and promote a sense of community among users.

Social network features 172 may be configured to extract information (e.g., health news and trends, best practices, etc.) from database 120 and share it with users via the social networking features. Data and metadata relating to social network sessions may be stored in database 120 and available for AI (including virtual coach 160), practitioners, and/or users. Data obtained from social network use may allow analysis to determine user- and community-level patterns, trends, associations, and insights, as well as conclusions and solutions that can be provided to the users and practitioners.

In some examples, platform 100 includes an analytics interface 176 configured to facilitate analytics performed on the data in database 120. The analytics interface may be used by, e.g., health researchers and practitioners associated with an owner or operator of the platform, third-party customers, and/or any other suitable parties.

For some parties (e.g., third-party customers), the analytics interface provides access only to anonymous and/or aggregated data, to protect the privacy of platform users and practitioners. Suitable third-party customers may include, e.g., health professionals and institutions, governments, universities, and businesses.

Data relating to use of analytics interface 176 (e.g., transactional data) is stored in database 120. Suitable data may include, e.g., search results of one or more customers, frequency of usage of one or more customers, etc. Metadata relating to use of the analytics interface (e.g., time spent on the site, location of user while using interface, etc.) is also captured and stored in the database.

### B. Illustrative Data Architecture

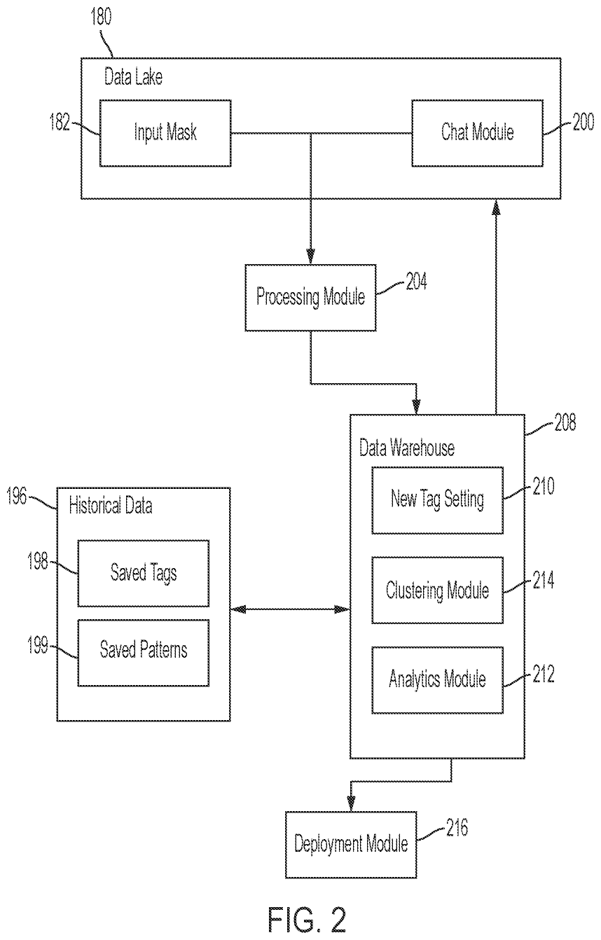

As shown in FIGS. 2-5, this section describes an illustrative machine learning (ML) based virtual practitioner system. This system is an example of a system suitable for use in the platform described above with respect to FIG. 1. FIGS. 2-5 depict the virtual practitioner system from a data flow and modularity perspective.

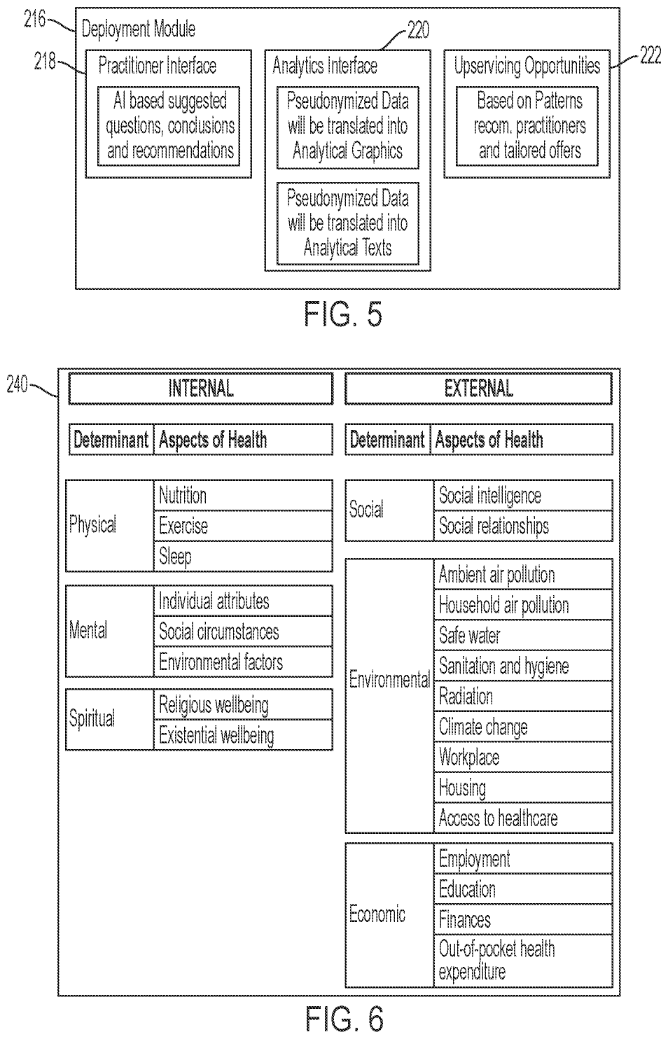

As described above, aspects of the present disclosure relate to the use of machine learning to create authentic virtual practitioner sessions based on a chatbot technology that evolves over time and is personalized to the user based on learned information. In general, systems and methods of the present disclosure include one or more of the features below:
Questions, conclusions, and recommendations for users, suggested by artificial intelligence (AI) functionality.
Questions, conclusions, and recommendations for practitioners, suggested by artificial intelligence (AI) functionality.
Translation of pseudonymized data into analytical graphics and text, presented in an analytics interface.
Upservicing opportunities based on patterns that can recommend certain practitioners and tailored offers.
See, e.g., FIG. 5.

Some features described above will be referred to again below in the context of the data flow, with the understanding that descriptions of such features throughout this disclosure are supplemental to each other and should be taken together.

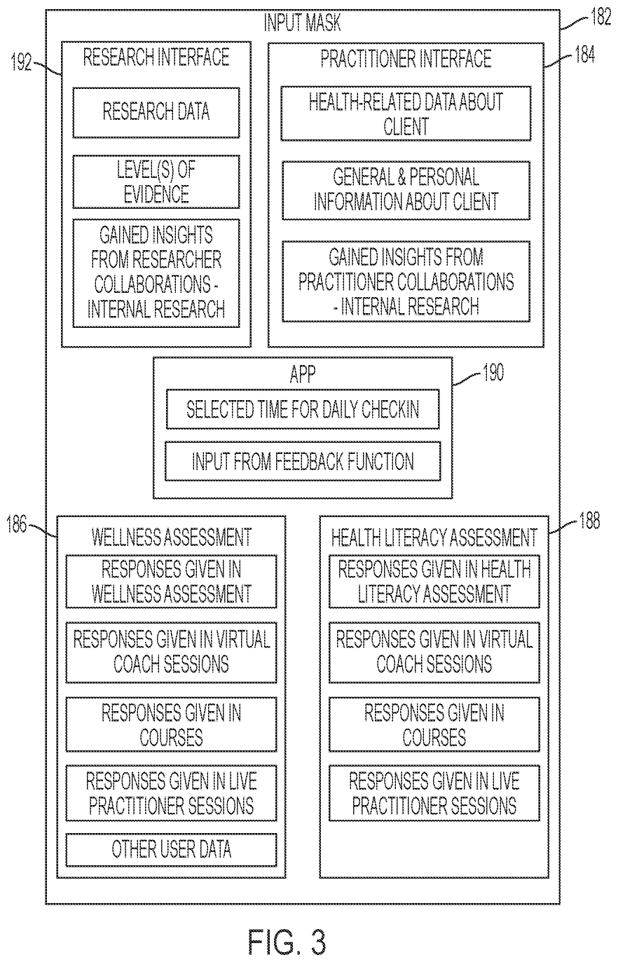

FIG. 2 is a schematic depiction of data flow of the virtual practitioner system. With reference to FIG. 2, data is collected from various sources and stored in a data lake 180. An input mask 182 includes a plurality of user interfaces and/or other features configured to collect data to be stored in data lake 180. As shown in FIG. 3, input mask 182 includes a practitioner interface 184 configured to facilitate input by health practitioners of data about the clients such as health-related data, general and personal information, and insights about the client gained from practitioner collaborations and research. Personal health data may include information such as allergies, previous and current symptoms and diseases, medications, diagnostics given by practitioners, etc. Demographic data can be included, such as location, gender, age. Data about lifestyle and family history may provide the practitioner with background information. Gained insights from practitioner collaborations and internal research can lead to relevant insights.

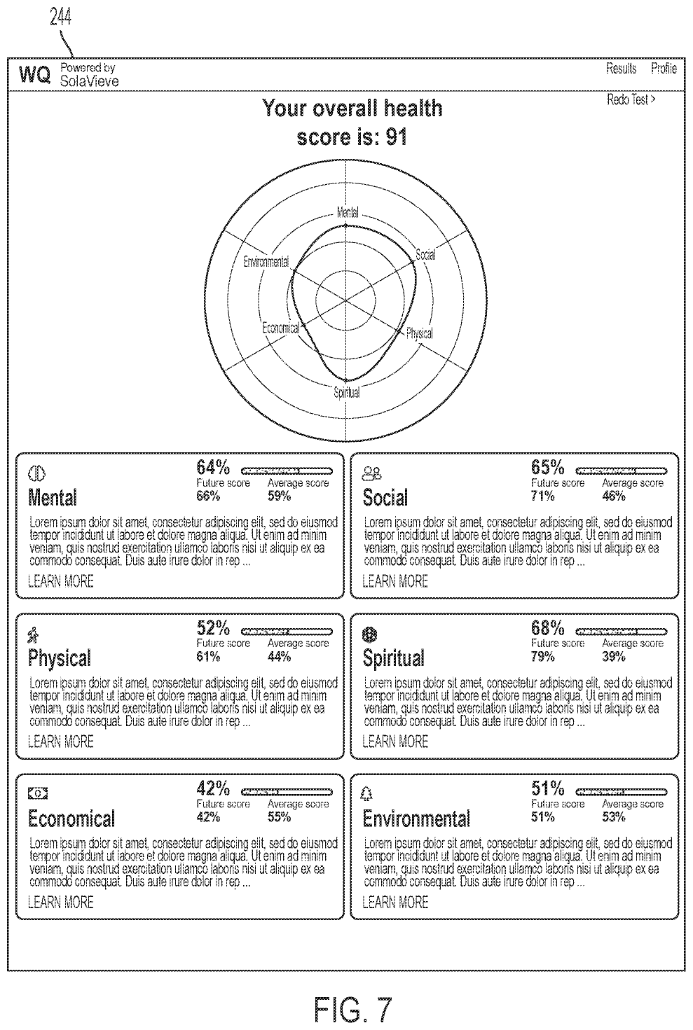

Input mask 182 further includes a wellbeing assessment 186 and a health literacy assessment 188. Wellbeing assessment 186 can comprise any suitable feature(s) configured to assess a user's wellbeing (e.g., one or more questions and/or other prompts in response to which a user can input response(s)). Health literacy assessment 188 can comprise any suitable feature(s) configured to assess a user's understanding of one or more aspects of health (e.g., questions and/or prompts relating to health and wellness in general and/or to a user's own health and/or habits). Examples of wellbeing and health literacy assessments are discussed below with reference to FIGS. 6-7. In some examples, one or more other suitable assessments are included in addition to, or instead of, a wellbeing and/or health literacy assessment.

Responses obtained via the wellbeing and health literacy assessments are also stored in data lake 180 in relation to the individual clients and in the aggregate. An overall wellbeing quotient of each individual user can be split into each of the six dimensions to get a more accurate picture of his or her current wellbeing in each of the dimensions. Likewise, an overall health literacy quotient of each individual user can be split into level and representative parameters of health literacy, as well as within each of the six dimensions to get a more accurate picture of his or her health literacy. See FIGS. 6-7.

App data 190 relating to the user app is also stored in data lake 180. App data 190 may include settings for time of periodic check-ins, input from the feedback function, and/or any other suitable data.

A research interface 192 is configured to facilitate input of research-related data (e.g., for inclusion in one or more knowledgebases), such as research findings; evidence and/or assessments of quality of evidence associated with the research findings; and insights based on research findings and/or on data associated with users of the platform (e.g., insights derived by an AI module of the platform). Research findings may include, e.g., health and wellness facts and/or statistics; findings on risk factors of various diseases and/or conditions; outcomes of scientific studies (e.g., on diseases, conditions, disease prevention, healthy lifestyles, etc.); documentation on best practices for practitioners; suitable questions for practitioners to ask practitioners in a consultation (or vice versa); symptom triage questions; diagnostic algorithms; and/or any other suitable research data on any suitable aspect of health and/or wellbeing. Research data may include data performed by third parties and/or by entities affiliated with the platform.

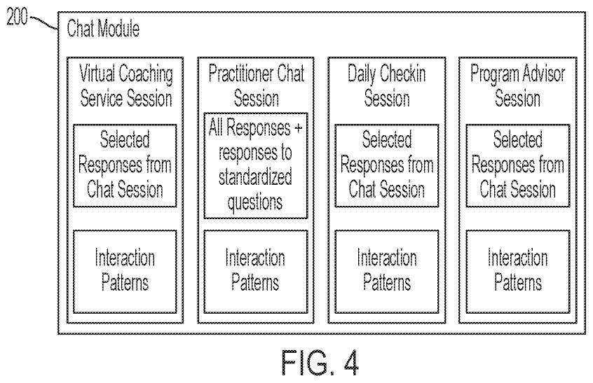

In addition to the above data sources, chat-related information is also stored in data lake 180. Chat-related information is received via a chat module 200 configured to facilitate chat-based user interaction with the platform. As shown in FIG. 4, in this example chat module 200 is configured to facilitate chat sessions with a virtual coaching service; with health practitioner(s); recurring (e.g., daily) check-in sessions with a virtual coach, practitioner, and/or automated system; and program advising sessions in which a virtual coach, practitioner, staff member, and/or automated system advises a user on a selection of programs (e.g., educational courses) offered by the platform.

Chat module 200 is configured to collect chat-related information, data relating to users' pattern of interaction with the chat sessions, messages provided to users in the chat sessions, and/or any other suitable information associated with chat sessions. Chat-related information may include client responses provided during chat sessions such as virtual coaching, practitioner chat, daily/weekly check-ins, program advisor sessions, and/or the like. Pattern-related data may include, for example, how long, how often, or how quickly the user interacts with a chat-related feature. Generally, users interact with practitioners in chat sessions based on free texts rather than selected rule-based answers.

As shown in FIG. 2, data stored in the data lake (e.g., the data described above) is passed on to a processing module 204. The processing module is configured to perform one or more functions with respect to preparing the data for storage, such as data selection, cleansing, extraction, input, transformation, storage, etc. Data cleansing, for example, includes fixing data by removing incorrect, corrupted, incorrectly formatted, duplicated, or incomplete data within a dataset.

After processing, the data is forwarded to a data warehouse 208, where new tag settings are determined by a new tag setting module 210. An analytics module 212 and a clustering module 214 are employed to perform data analytics and clustering. The data is also compared against a store of historical data 196, which may include saved tags 198, patterns 199, and/or any other suitable historical data.

A deployment module 216 accesses the data from the data warehouse and provides interfaces for users (AKA user interfaces or UIs). FIG. 5 depicts an example of deployment module 216. As shown in the example of FIG. 5, a Practitioner UI 218 is configured to provide questions, recommendations, and conclusions (e.g., as suggested questions, recommendations, and/or conclusions that the practitioner may pose to a user). Aggregated and analyzed data is presented via an Analytics UI 220, visually and/or textually. An Upservicing UI 222 is configured to present upservicing opportunities based on, e.g., patterns observed in a user's interactions with the platform. For example, the Upservicing UI may present recommended practitioners and/or tailored offerings.

### C. Illustrative Wellbeing Assessment

With reference to FIG. 6, this section describes an illustrative wellbeing assessment 240 configured to assess various aspects of a user's health. Wellbeing assessment 240 is an example of wellbeing assessment 186 described above with reference to FIG. 3. The wellbeing assessment may be administered to a user by a chat module and/or other suitable portion of platform 100 and/or another suitable platform, as described above.

The wellbeing assessment is generally configured to assess a user's health from a holistic perspective, accounting for the complexity and interconnectedness of an individual's health and wellbeing.

The wellbeing assessment of this example comprises a plurality of questions presentable to users and configured to evaluate user responses to assess an array of internal and external health determinants. See FIG. 6. The internal and external health determinants are further subdivided into a plurality of aspects of health, with FIG. 6 providing a non-exhaustive list of aspects. The assessment's questions are distributed suitably (e.g., uniformly, and/or in any other suitable manner) among the determinants, with some questions effecting multiple determinants.

The wellbeing assessment is configured to assess a user's lifestyle by considering, e.g., the following elements:
1. Behaviors with short-term impact on health
2. Behaviors with long-term impact on health
3. Access to health-related services, means, and infrastructures

Accordingly, the questions and/or prompts presented to the user by the wellbeing assessment comprise three parts. In the first part (Part One), the assessment asks a series of questions on the user's “current” health status. Based on the user's responses, the system (e.g., platform 100) formulates an overall score (the wellbeing score, which like the IQ score is from 0 to 160). The system also calculates sub-scores for each health dimension and each sub-aspect of a dimension. These scores range from 0% to 100%.

In the second part (Part Two), the assessment asks a series of “future” oriented questions about the user's willingness to change behavior and lifestyle to improve their health. The user's scores from Part One determine which “future” oriented questions are asked. The lower the “current” score on a question, the higher the chance of being asked a corresponding “future” oriented question, because a low “current” score tends to indicate a poor area of health. In this example, every current question from Part One has a corresponding future question in Part Two. Based on the user's “future” responses, the system calculates the “future” scores for the overall wellbeing, health dimensions and sub-aspects.

In the third part (Part Three), the assessment asks the user which health dimension is most meaningful for them to address.

In some examples, other suitable user data is factored into the wellbeing assessment. For example, data collected from a user's wearable device(s), and/or any other suitable user data, may be included in the wellbeing assessment (e.g., may at least partially determine one or more of the user's scores on the assessment and/or other output presented to the user based on the assessment). In general, any suitable user data described herein can be incorporated into the wellbeing assessment.

After taking the assessment, the wellbeing assessment presents to the user a series of numeric scores:
Their “current” scores for the overall wellbeing, each health dimension, and the sub-aspects
Their “future” scores for the overall wellbeing, each health dimension, and the sub-aspects
The “average” scores (same set of scores as noted above) of all the users who have already taken the test, to provide benchmarking.
The “predictive” scores for the overall wellbeing, each health dimension, and the sub-aspects. Whereas the “future” score is derived from the user's input on “future” questions, the “predictive” score provides the user a prediction on their future health status based on data mining of their total set of user data, as well as predictive modeling, machine learning techniques and algorithms.

The “predictive population” scores for the overall wellbeing, each health dimension, and the sub-aspects. The “predictive population” score provides a prediction on the future health status of a specified population of people (e.g., by gender, age, nationality, pre-defined cohort (such as employees of a company), etc.), based on anonymous data mining of the total set of population data, as well as predictive modeling, machine learning techniques and algorithms. The wellbeing assessment may additionally present a customized narrative report for the user based specifically on their scores, as well as recommendations based on the assessment results. Suitable recommendations may include, e.g., which services and/or education courses of the platform a user may benefit from, general healthy lifestyle actions based on their test results, and/or any other suitable recommendations. FIG. 7 depicts an illustrative report 244 (presented to a user, e.g., via an app, website, and/or other suitable feature of the platform) including a user's numeric scores along six health dimensions.

A report based on the wellbeing assessment may, e.g., describe strengths and/or weaknesses of a user, and suggest ways in which a user has the ability to make positive change in their wellbeing. In some examples, the report suggests ways in which strengths demonstrated by a user in some areas of their life may be transferable to weaker areas.

For some users, the wellbeing assessment creates a baseline understanding of their health status. Additionally, or alternatively, it may be retaken by the user frequently to assess how their health is progressing or digressing.

Questions comprising the wellbeing assessment and algorithm(s) configured to score the wellbeing assessment are stored in the platform's central database (e.g., database 120 of platform 100). The questions and/or algorithm used in a given situation may be dynamically adjusted based on certain factors, such as user gender, country of residence, and/or any other suitable factor. Wellbeing questions can be administered throughout platform 100—for example, in an assessment tool, inside AI virtual coaching sessions, during live practitioner sessions, etc. All suitable user data collected is aggregated, analyzed, and calculated to derive scores for the wellbeing. Elements and related algorithms of the wellbeing are ever-growing and enhancing as new research is identified and content developed.

User responses to the wellbeing assessment are added to database 120 (and/or another suitable database of another suitable platform). Metadata (e.g., time of day when the assessment is taken, location where the test is taken, type of device used to take the test, the time taken by a user to answer one or more questions or groups of questions) may also be added to the database. Actions taken by a user (e.g., signing up for a platform service or course, accessing information in the database, etc.) after taking the assessment (and/or after reading their results) may also be added to the database.

In some examples, a wellbeing assessment and/or other suitable assessment may be devised in a manner that takes into account at least some aspects of Solution-Focused Brief Therapy methodology, which assumes that the individual possesses a motivated, active willingness to make lifestyle changes to improve their health and wellbeing. In these examples, the assessment provides individuals with the opportunity to notice and reflect on their recent lifestyle choices and the areas of their life where they feel confident and possess strengths. The assessment may prompt a user to go deeper by assessing their willingness to make positive behavior change specifically in the areas where they are reported to be the weakest—leveraging their strengths to empower them, demonstrate their capabilities, and increase their confidence in adopting new behaviors. Both strategies enable the assessment to estimate a current health state and a desired future health state. From there, the assessment asks the individual which health determinant, if improved, would make the biggest impact on their life. This can be a key question, as it establishes a starting point for effective behavioral change.

### D. Illustrative Health Literacy Assessment

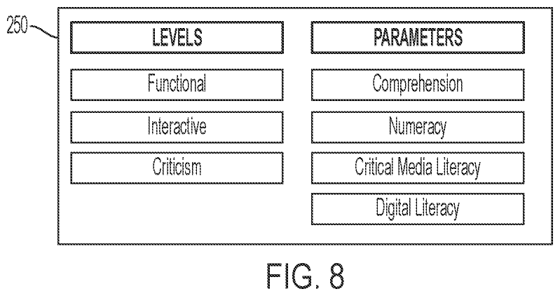

With reference to FIG. 8, this section describes an illustrative health literacy assessment 250 configured to assess various levels, parameters, and health dimensions and aspects of a user's health literacy. The health literacy assessment may be administered to a user by a chat module and/or other suitable portion of platform 100 and/or another suitable platform, as described above.

The health literacy assessment is generally configured to assess a user's knowledge of health information, their awareness of their own health status and lifestyle, and the gap between knowledge and actions from a health from a holistic perspective, accounting for the complexity and interconnectedness of an individual's health and wellbeing.

The health literacy assessment of this example comprises a plurality of questions presentable to users and configured to evaluate user responses to assess an array of degrees and parameters. See FIG. 8. The levels and parameters are further subdivided into a plurality of aspects of health, with FIG. 8 providing a non-exhaustive list of aspects. The assessment's questions are distributed suitably (e.g., uniformly, and/or in any other suitable manner) among the levels and parameters, with some questions effecting multiple levels and/or multiple parameters.

The health literacy assessment is configured to assess a user's health knowledge lifestyle by considering, e.g., the following degrees:

1. Functional health literacy or know-why refers to the effective communication of information.

2. Interactive or know-how talks about the possibility of acquiring new skills. This can be also considered as the ability to know and understand one's own health status, as well as their goals, intentions, strengths, challenges, lifestyle habits, patterns, etc.

3. Criticism or application includes the empowerment of a person and the surrounding community regarding healthy living, and further includes the knowing-doing gap; that is, the difference between knowing what to do to achieve sustainable health and actually doing it.

These degrees are further subdivided by the parameters (e.g., health dimensions and aspects) shown in FIG. 8. Individual assessment elements contained within the degrees, health dimensions and aspects are configured to assess a user's health knowledge by considering, e.g., the following representative parameters of health literacy elements:

1. Comprehension: the capacity to understand health-related content in terms of reading ability.

2. Numeracy: the degree to which individuals can access, process, interpret, communicate, and act on numerical, quantitative, graphical, and probabilistic health information needed to make effective health decisions. It is not simply understanding (processing and interpreting), but also communicating and acting according to numeric terms. This entails the ability to understand food labels, measuring medication, and interpreting physical parameters, such as weight, blood pressure, blood glucose, and understanding risks. A lack of numeracy is associated with the inability to make informed comparisons using numbers, a lack of trust in information that contains numbers, and being more influenced by a trusted source over numerical information.

3. Critical media literacy: the ability to analyze information for credibility, purpose, and quality.

4. Digital literacy: the ability to appropriately use digital tools to identify, access, manage, analyze, and synthesize digital resources.

The health literacy assessment may ask a series of questions related to the degrees, parameters, health dimensions, and aspects.

Weighted scoring may be applied to the health literacy degrees, health dimensions, aspects, and parameters. Based on the user's responses, the system (e.g., platform 100) formulates an overall score (the health literacy score, which ranges from 0% to 100%) within tiers of levels. Levels range from 0-10, with 10 demonstrating the highest level of health literacy. Each level contains a curriculum of knowledge to achieve before progressing to the next level. Continuous education will accompany Level 10 graduates ad infinitum to ensure the individual's knowledge base is kept up to date and includes the latest research, insights, and findings. The system also calculates sub-scores for each health dimension and each sub-aspect of a dimension. These scores range from 0% to 100% within the level tier system.

Health literacy assessments may be provided all at once or over a period of time. After taking the assessment, a health literacy report is presented to the user through a series of numeric scores and levels, presenting overall scoring and sub-divided scoring.

The health literacy assessment may additionally present a customized narrative report for the user based specifically on their scores, as well as recommendations based on the assessment results. Suitable recommendations may include, e.g., which services and/or education courses of the platform a user may benefit from, general healthy lifestyle actions based on their test results, and/or any other suitable recommendations.

A report based on the health literacy assessment may, e.g., describe strengths and/or weaknesses of a user, and suggest ways in which a user has the ability to make positive change in their health literacy and wellbeing. In some examples, the report suggests ways in which strengths demonstrated by a user in some areas of their life may be transferable to weaker areas.

For some users, the health literacy assessment creates a baseline understanding of their health knowledge. Additionally, or alternatively, it may be retaken by the user frequently to assess how their health knowledge is progressing or digressing.

Questions comprising the health literacy assessment and algorithm(s) configured to score the health literacy assessment are stored in the platform's central database (e.g., database 120 of platform 100). The questions and/or algorithm used in a given situation may be dynamically adjusted based on certain factors, such as user gender, country of residence, and/or any other suitable factor. Health literacy questions can be administered throughout platform 100—for example, in an assessment tool, inside AI virtual coaching sessions, during live practitioner sessions, etc. All suitable user data collected is aggregated, analyzed, and calculated to derive scores for health literacy. Elements and related algorithms of the health literacy are ever-growing and enhancing as new research is identified and content developed.

User responses to the health literacy assessment are added to database 120 (and/or another suitable database of another suitable platform). Metadata (e.g., time of day when the assessment is taken, location where the test is taken, type of device used to take the test, the time taken by a user to answer one or more questions or groups of questions) may also be added to the database. Actions taken by a user (e.g., signing up for a platform service or course, accessing information in the database, etc.) after taking the assessment (and/or after reading their results) may also be added to the database.

In some examples, a health literacy assessment and/or other suitable assessment may be devised in a manner that takes into account at least some aspects of Solution-Focused Brief Therapy methodology, which assumes that the individual possesses a motivated, active willingness to make lifestyle changes to improve their health and wellbeing. In these examples, the assessment provides individuals with the opportunity to notice and reflect on their recent lifestyle choices and the areas of their life where they feel confident and possess strengths. The assessment may prompt a user to go deeper by assessing their willingness to make positive behavior change specifically in the areas where they are reported to be the weakest—leveraging their strengths to empower them, demonstrate their capabilities, and increase their confidence in adopting new behaviors. Both strategies enable the assessment to estimate aspects of health literacy.

### E. Illustrative App

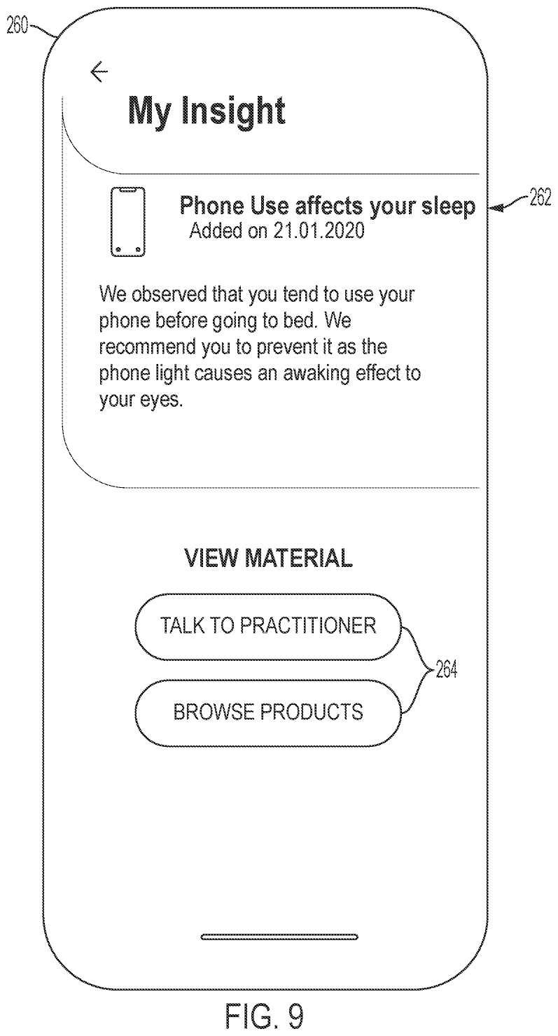

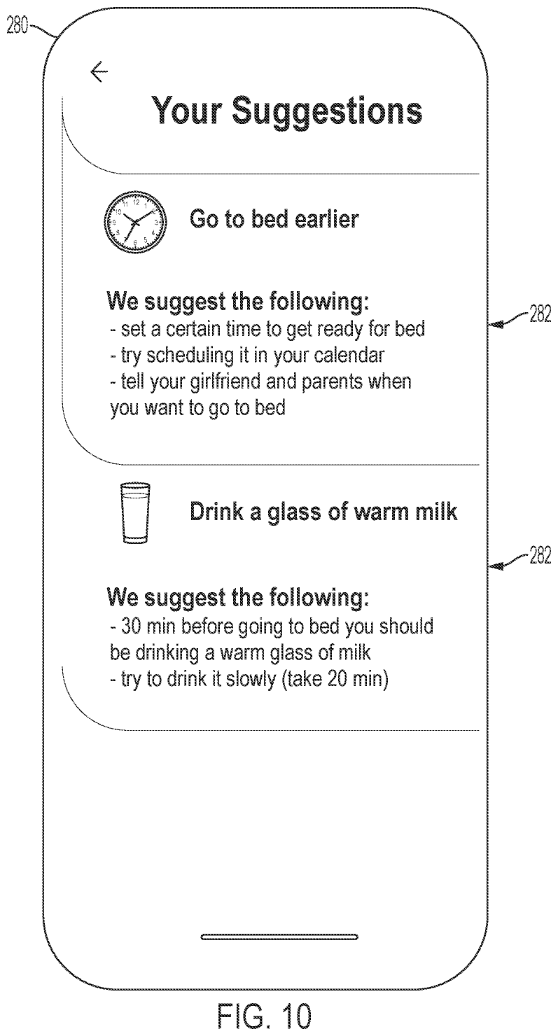

With reference to FIGS. 9-10, this section describes aspects of an illustrative software app configured to facilitate user interaction with a digital health and wellness platform such as platform 100.

FIG. 9 depicts a screenshot 260 in which the app presents to the user an insight 262 derived by the platform. In this example, the insight includes an observation that the user frequently uses their smartphone before going to bed. Using a smartphone or similar device shortly before going to sleep is known to have potential to adversely affect sleep quality. Accordingly, in screenshot 260, the app presents a recommendation that the user stop using their smartphone around bedtime. The app presents users with buttons 264 clickable to view further educational material (e.g., about sleep quality and/or the effects of electronics use on sleep quality), to talk to a practitioner, and/or to browse products for purchase.

Insight 262 is an example of an insight derived by the platform (e.g., by an AI module of the platform) based on user interaction data (in this example, a user's pattern of frequently using their smartphone around bedtime). Insight 262 may be one of a plurality of different insights produced by the platform and presented to the user. These insights may be developed based on user interaction data, user responses to chat sessions, and/or based on any other suitable data in any suitable manner, as described elsewhere herein.

In this example, the insight comprises an observation, and the app presents a recommendation to the user based on that insight. In other examples, the platform (e.g., an AI module of the platform) is configured to derive a recommendation based on data of the platform (e.g., user interaction data and/or any other suitable data) rather than first deriving an observation and deriving a recommendation based on the observation. Put another way, the insight derived by the platform may comprise a recommendation rather than an observation, or in addition to an observation.

FIG. 10 depicts a screenshot 280 in which the app presents to the user a plurality of suggestions 282 the user may wish to adopt to improve their health and wellbeing. Each of the suggestions may be provided by a practitioner, by a researcher, and/or derived automatically by the platform (e.g., an AI module of the platform) based on user data, based on user assessment responses and/or scores, based on platform-generated insights (e.g., observations and/or recommendations), and/or generated in any other suitable manner.

### F. Illustrative Method

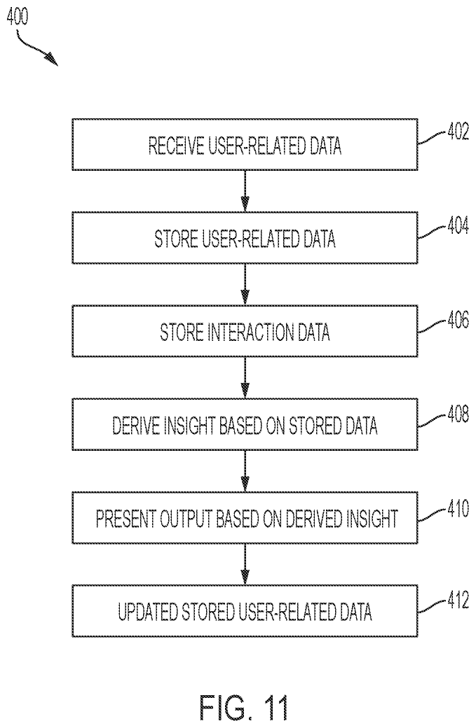

With reference to FIG. 11, this section describes steps of an illustrative method 400 for providing digital health and wellness services. Aspects of platform 100 may be utilized in the method steps described below. Where appropriate, reference may be made to components and systems that may be used in carrying out each step. These references are for illustration, and are not intended to limit the possible ways of carrying out any particular step of the method.

FIG. 11 is a flowchart illustrating steps performed in an illustrative method, and may not recite the complete process or all steps of the method. Although various steps of method 400 are described below and depicted in FIG. 11, the steps need not necessarily all be performed, and in some cases may be performed simultaneously or in a different order than the order shown.

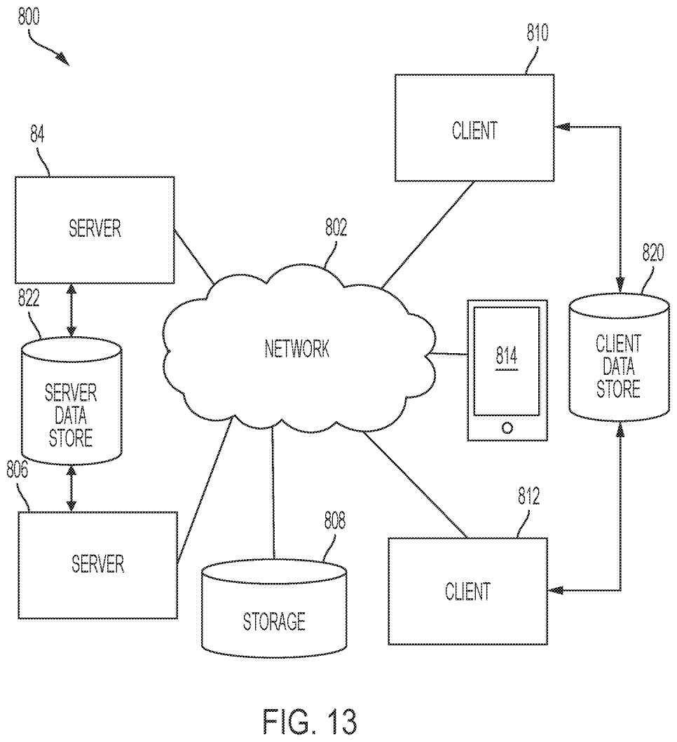

Step 402 of method 400 optionally includes receiving, at a data processing system of a digital health and wellness platform, data relating to a user. Step 404 of method 400 includes storing the user-related data (e.g., at a memory store of the data processing system of the platform). In some examples, the user-related data is input by a user using a client device (e.g., a computer, smartphone, and/or other suitable device) accessing the platform via a website, app, and/or the like. Accordingly, in these examples, step 402 includes receiving the user-related data at the data processing system, which may comprise a server, via a communications network (see, e.g., FIG. 13 and associated description below).

In some examples, step 402 includes facilitating a chat session between the user and a health practitioner who is located remotely from the user and using another client device to access the platform via a website, app, and/or the like (e.g., using a practitioner interface of the platform). Alternatively, or additionally, step 402 may include facilitating a chat session between the user and a virtual coach comprising one or more AI systems. Chat messages provided by the user, or provided to the user by the practitioner or virtual coach, can be stored by the platform. Alternatively, or additionally, step 402 may include receiving user input in response to one or more prompts outside of a chat session, such as questions comprising a wellbeing or health literacy assessment, and step 404 may include storing the received user input.

In some examples, the platform stores additional data, such as data relating to the user input by a health practitioner; aggregated user data and/or statistics based on aggregated user data; demographic data and/or statistics obtained from third-party sources; and/or any other suitable data described herein.

Step 406 of method 400 optionally includes storing data relating to a user's interaction with the platform (e.g., storing the interaction data as part of the user-related data). The interaction data may include and/or be derived from metadata associated with user input, the presentation of platform content to the user, and/or any other suitable user interaction. For example, interaction data may include information on geographic locations and/or times of day at which the user accesses the platform or has certain interactions with the platform, durations of user interactions with the platform, frequency of user interactions with the platform, and/or any other suitable interaction data.

Step 408 of method 400 includes deriving an insight based on at least a portion of the stored data. An insight may be derived based on the data by any suitable process(es), including machine learning, natural language processing, and/or any other suitable form of artificial intelligence; statistical analysis; a rule-based (e.g., non-machine-learning) system; and/or any other suitable method of deriving an insight based on data. In some examples, the insight is derived by the virtual coach during or after a chat session between the user and the virtual coach.

In some examples, an insight comprises an observation, conclusion, trend, pattern, and/or statistic determined based on the data. For example, an insight may include an observation that a user is presently in a sad mood, or that a user repeatedly experiences a same emotion or mood at a certain time of day or following a certain type of event, experiences a pattern of sleeping poorly following certain behavior, experiences good health following certain behavior or events, and/or any other suitable observations.

Alternatively, or additionally, an insight may comprise a recommended course of action that a user can take to improve their wellness. For example, an insight may include a recommendation that a user get more sleep, have a chat session with a health practitioner, complete a course on the platform, and/or take any other suitable action. In some examples, an insight comprises an observation and an associated recommended action (e.g., the insight may comprise an observation that a user is getting insufficient sleep and a recommendation that the user go to bed earlier).

In some examples wherein an insight comprises an observation and does not include a recommendation, a recommendation may be derived based on the insight using artificial intelligence, rule-based systems, and/or any other suitable method(s).

Step 410 of method 400 includes presenting output to the user based on the derived insight. The output may include, e.g., information expressing and/or explaining the insight, a recommendation based on the insight (e.g., in examples wherein the insight does not itself include a recommendation), and/or any other suitable content. The output may have any suitable form, such as visual (e.g., text, graphics, animations, etc.), auditory, haptic, etc. In some examples, the insight of step 408 is derived during a chat session with a virtual coach, and presenting output based on the insight at step 410 includes automatically determining a response for the virtual coach to provide to the user based on the insight and presenting the determined response to the user as a chat response from the virtual coach. For example, the virtual coach may express the insight to the user in a chat message and/or may automatically determine, based on the insight, a question to ask the user, a type of language or vocabulary or syntax with which to express a message, and/or any other suitable aspect(s) of the virtual coach chat session.

Step 412 of method 400 optionally includes updating the stored user data based on the derived insight and/or on the output presented to the user. For example, insights determined for a user may be stored along with other data related to that user, and future insights may be determined based at least partially on the stored insight. As another example, a health practitioner conducting a chat or video session with the user may consult the stored user data and see the stored insight and/or recommendations previously made to the user, which may inform any questions or recommendations posed by the practitioner to the user in the current session. In some examples, the stored user data is used to train machine-learning or other artificially intelligent aspects of the platform, such as the virtual coach. In general, the stored insights and/or recommendations may be used in any suitable manner for using user-related data as described herein.

### G. Illustrative Data Processing System

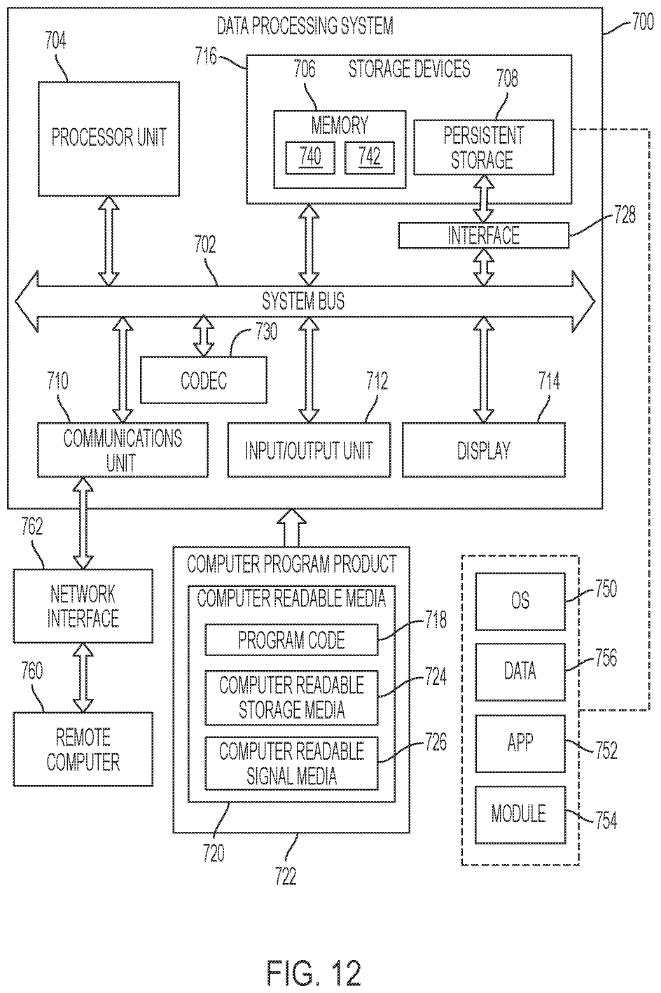

As shown in FIG. 12, this example describes a data processing system 700 (also referred to as a computer, computing system, and/or computer system) in accordance with aspects of the present disclosure. In this example, data processing system 700 is an illustrative data processing system suitable for implementing aspects of the digital health platform. More specifically, in some examples, devices that are embodiments of data processing systems (e.g., smartphones, tablets, personal computers) may be used by users, practitioners, researchers, and/or any other suitable parties to access the platform (e.g., via a PWA, standalone application, and/or any other suitable implementation). Additionally, or alternatively, aspects of the platform performing artificial intelligence and/or analytics (e.g., the virtual coach, analytics features, and/or any other suitable parts of the platform) may be implemented on one or more data-processing systems.

In this illustrative example, data processing system 700 includes a system bus 702 (also referred to as communications framework). System bus 702 may provide communications between a processor unit 704 (also referred to as a processor or processors), a memory 706, a persistent storage 708, a communications unit 710, an input/output (I/O) unit 712, a codec 730, and/or a display 714. Memory 706, persistent storage 708, communications unit 710, input/output (I/O) unit 712, display 714, and codec 730 are examples of resources that may be accessible by processor unit 704 via system bus 702.

Processor unit 704 serves to run instructions that may be loaded into memory 706. Processor unit 704 may comprise a number of processors, a multi-processor core, and/or a particular type of processor or processors (e.g., a central processing unit (CPU), graphics processing unit (GPU), etc.), depending on the particular implementation. Further, processor unit 704 may be implemented using a number of heterogeneous processor systems in which a main processor is present with secondary processors on a single chip. As another illustrative example, processor unit 704 may be a symmetric multi-processor system containing multiple processors of the same type.

Memory 706 and persistent storage 708 are examples of storage devices 716. A storage device may include any suitable hardware capable of storing information (e.g., digital information), such as data, program code in functional form, and/or other suitable information, either on a temporary basis or a permanent basis.

Storage devices 716 also may be referred to as computer-readable storage devices or computer-readable media. Memory 706 may include a volatile storage memory 740 and a non-volatile memory 742. In some examples, a basic input/output system (BIOS), containing the basic routines to transfer information between elements within the data processing system 700, such as during start-up, may be stored in non-volatile memory 742. Persistent storage 708 may take various forms, depending on the particular implementation.

Persistent storage 708 may contain one or more components or devices. For example, persistent storage 708 may include one or more devices such as a magnetic disk drive (also referred to as a hard disk drive or HDD), solid state disk (SSD), floppy disk drive, tape drive, Jaz drive, Zip drive, flash memory card, memory stick, and/or the like, or any combination of these. One or more of these devices may be removable and/or portable, e.g., a removable hard drive. Persistent storage 708 may include one or more storage media separately or in combination with other storage media, including an optical disk drive such as a compact disk ROM device (CD-ROM), CD recordable drive (CD-R Drive), CD rewritable drive (CD-RW Drive), and/or a digital versatile disk ROM drive (DVD-ROM). To facilitate connection of the persistent storage devices 708 to system bus 702, a removable or non-removable interface is typically used, such as interface 728.

Input/output (I/O) unit 712 allows for input and output of data with other devices that may be connected to data processing system 700 (i.e., input devices and output devices). For example, an input device may include one or more pointing and/or information-input devices such as a keyboard, a mouse, a trackball, stylus, touch pad or touch screen, microphone, joystick, game pad, satellite dish, scanner, TV tuner card, digital camera, digital video camera, web camera, and/or the like. These and other input devices may connect to processor unit 704 through system bus 702 via interface port(s). Suitable interface port(s) may include, for example, a serial port, a parallel port, a game port, and/or a universal serial bus (USB).

One or more output devices may use some of the same types of ports, and in some cases the same actual ports, as the input device(s). For example, a USB port may be used to provide input to data processing system 700 and to output information from data processing system 700 to an output device. One or more output adapters may be provided for certain output devices (e.g., monitors, speakers, and printers, among others) which require special adapters. Suitable output adapters may include, e.g. video and sound cards that provide a means of connection between the output device and system bus 702. Other devices and/or systems of devices may provide both input and output capabilities, such as remote computer(s) 760. Display 714 may include any suitable human-machine interface or other mechanism configured to display information to a user, e.g., a CRT, LED, or LCD monitor or screen, etc.

Communications unit 710 refers to any suitable hardware and/or software employed to provide for communications with other data processing systems or devices. While communication unit 710 is shown inside data processing system 700, it may in some examples be at least partially external to data processing system 700. Communications unit 710 may include internal and external technologies, e.g., modems (including regular telephone grade modems, cable modems, and DSL modems), ISDN adapters, and/or wired and wireless Ethernet cards, hubs, routers, etc. Data processing system 700 may operate in a networked environment, using logical connections to one or more remote computers 760. A remote computer(s) 760 may include a personal computer (PC), a server, a router, a network PC, a workstation, a microprocessor-based appliance, a peer device, a smart phone, a tablet, another network note, and/or the like. Remote computer(s) 760 typically include many of the elements described relative to data processing system 700. Remote computer(s) 760 may be logically connected to data processing system 700 through a network interface 762 which is connected to data processing system 700 via communications unit 710. Network interface 762 encompasses wired and/or wireless communication networks, such as local-area networks (LAN), wide-area networks (WAN), and cellular networks. LAN technologies may include Fiber Distributed Data Interface (FDDI), Copper Distributed Data Interface (CDDI), Ethernet, Token Ring, and/or the like. WAN technologies include point-to-point links, circuit switching networks (e.g., Integrated Services Digital networks (ISDN) and variations thereon), packet switching networks, and Digital Subscriber Lines (DSL).

Codec 730 may include an encoder, a decoder, or both, comprising hardware, software, or a combination of hardware and software. Codec 730 may include any suitable device and/or software configured to encode, compress, and/or encrypt a data stream or signal for transmission and storage, and to decode the data stream or signal by decoding, decompressing, and/or decrypting the data stream or signal (e.g., for playback or editing of a video). Although codec 730 is depicted as a separate component, codec 730 may be contained or implemented in memory, e.g., non-volatile memory 742.

Non-volatile memory 742 may include read only memory (ROM), programmable ROM (PROM), electrically programmable ROM (EPROM), electrically erasable programmable ROM (EEPROM), flash memory, and/or the like, or any combination of these. Volatile memory 740 may include random access memory (RAM), which may act as external cache memory. RAM may comprise static RAM (SRAM), dynamic RAM (DRAM), synchronous DRAM (SDRAM), double data rate SDRAM (DDR SDRAM), enhanced SDRAM (ESDRAM), and/or the like, or any combination of these.

Instructions for the operating system, applications, and/or programs may be located in storage devices 716, which are in communication with processor unit 704 through system bus 702. In these illustrative examples, the instructions are in a functional form in persistent storage 708. These instructions may be loaded into memory 706 for execution by processor unit 704. Processes of one or more embodiments of the present disclosure may be performed by processor unit 704 using computer-implemented instructions, which may be located in a memory, such as memory 706.

These instructions are referred to as program instructions, program code, computer usable program code, or computer-readable program code executed by a processor in processor unit 704. The program code in the different embodiments may be embodied on different physical or computer-readable storage media, such as memory 706 or persistent storage 708. Program code 718 may be located in a functional form on computer-readable media 720 that is selectively removable and may be loaded onto or transferred to data processing system 700 for execution by processor unit 704. Program code 718 and computer-readable media 720 form computer program product 722 in these examples. In one example, computer-readable media 720 may comprise computer-readable storage media 724 or computer-readable signal media 726.

Computer-readable storage media 724 may include, for example, an optical or magnetic disk that is inserted or placed into a drive or other device that is part of persistent storage 708 for transfer onto a storage device, such as a hard drive, that is part of persistent storage 708. Computer-readable storage media 724 also may take the form of a persistent storage, such as a hard drive, a thumb drive, or a flash memory, that is connected to data processing system 700. In some instances, computer-readable storage media 724 may not be removable from data processing system 700.

In these examples, computer-readable storage media 724 is a non-transitory, physical or tangible storage device used to store program code 718 rather than a medium that propagates or transmits program code 718. Computer-readable storage media 724 is also referred to as a computer-readable tangible storage device or a computer-readable physical storage device. In other words, computer-readable storage media 724 is media that can be touched by a person.

Alternatively, program code 718 may be transferred to data processing system 700, e.g., remotely over a network, using computer-readable signal media 726. Computer-readable signal media 726 may be, for example, a propagated data signal containing program code 718. For example, computer-readable signal media 726 may be an electromagnetic signal, an optical signal, and/or any other suitable type of signal. These signals may be transmitted over communications links, such as wireless communications links, optical fiber cable, coaxial cable, a wire, and/or any other suitable type of communications link. In other words, the communications link and/or the connection may be physical or wireless in the illustrative examples.

In some illustrative embodiments, program code 718 may be downloaded over a network to persistent storage 708 from another device or data processing system through computer-readable signal media 726 for use within data processing system 700. For instance, program code stored in a computer-readable storage medium in a server data processing system may be downloaded over a network from the server to data processing system 700. The computer providing program code 718 may be a server computer, a client computer, or some other device capable of storing and transmitting program code 718.

In some examples, program code 718 may comprise an operating system (OS) 750. Operating system 750, which may be stored on persistent storage 708, controls and allocates resources of data processing system 700. One or more applications 752 take advantage of the operating system's management of resources via program modules 754, and program data 756 stored on storage devices 716. OS 750 may include any suitable software system configured to manage and expose hardware resources of computer 700 for sharing and use by applications 752. In some examples, OS 750 provides application programming interfaces (APIs) that facilitate connection of different type of hardware and/or provide applications 752 access to hardware and OS services. In some examples, certain applications 752 may provide further services for use by other applications 752, e.g., as is the case with so-called “middleware.” Aspects of present disclosure may be implemented with respect to various operating systems or combinations of operating systems.

The different components illustrated for data processing system 700 are not meant to provide architectural limitations to the manner in which different embodiments may be implemented. One or more embodiments of the present disclosure may be implemented in a data processing system that includes fewer components or includes components in addition to and/or in place of those illustrated for computer 700. Other components shown in FIG. 12 can be varied from the examples depicted. Different embodiments may be implemented using any hardware device or system capable of running program code. As one example, data processing system 700 may include organic components integrated with inorganic components and/or may be comprised entirely of organic components (excluding a human being). For example, a storage device may be comprised of an organic semiconductor.

In some examples, processor unit 704 may take the form of a hardware unit having hardware circuits that are specifically manufactured or configured for a particular use, or to produce a particular outcome or progress. This type of hardware may perform operations without needing program code 718 to be loaded into a memory from a storage device to be configured to perform the operations. For example, processor unit 704 may be a circuit system, an application specific integrated circuit (ASIC), a programmable logic device, or some other suitable type of hardware configured (e.g., preconfigured or reconfigured) to perform a number of operations. With a programmable logic device, for example, the device is configured to perform the number of operations and may be reconfigured at a later time. Examples of programmable logic devices include, a programmable logic array, a field programmable logic array, a field programmable gate array (FPGA), and other suitable hardware devices. With this type of implementation, executable instructions (e.g., program code 718) may be implemented as hardware, e.g., by specifying an FPGA configuration using a hardware description language (HDL) and then using a resulting binary file to (re)configure the FPGA.

In another example, data processing system 700 may be implemented as an FPGA-based (or in some cases ASIC-based), dedicated-purpose set of state machines (e.g., Finite State Machines (FSM)), which may allow critical tasks to be isolated and run on custom hardware. Whereas a processor such as a CPU can be described as a shared-use, general purpose state machine that executes instructions provided to it, FPGA-based state machine(s) are constructed for a special purpose, and may execute hardware-coded logic without sharing resources. Such systems are often utilized for safety-related and mission-critical tasks.

In still another illustrative example, processor unit 704 may be implemented using a combination of processors found in computers and hardware units. Processor unit 704 may have a number of hardware units and a number of processors that are configured to run program code 718. With this depicted example, some of the processes may be implemented in the number of hardware units, while other processes may be implemented in the number of processors.

In another example, system bus 702 may comprise one or more buses, such as a system bus or an input/output bus. Of course, the bus system may be implemented using any suitable type of architecture that provides for a transfer of data between different components or devices attached to the bus system. System bus 702 may include several types of bus structure(s) including memory bus or memory controller, a peripheral bus or external bus, and/or a local bus using any variety of available bus architectures (e.g., Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI)).

Additionally, communications unit 710 may include a number of devices that transmit data, receive data, or both transmit and receive data. Communications unit 710 may be, for example, a modem or a network adapter, two network adapters, or some combination thereof. Further, a memory may be, for example, memory 706, or a cache, such as that found in an interface and memory controller hub that may be present in system bus 702.

### H. Illustrative Distributed Data Processing System

As shown in FIG. 13, this example describes a general network data processing system 800, interchangeably termed a computer network, a network system, a distributed data processing system, or a distributed network, aspects of which may be included in one or more illustrative embodiments of digital health platforms. For example, users, practitioners, researchers, and/or other parties may use one or more networks to access the platform. As another example, different aspects of the platform may communicate with each other using one or more networks.

It should be appreciated that FIG. 13 is provided as an illustration of one implementation and is not intended to imply any limitation with regard to environments in which different embodiments may be implemented. Many modifications to the depicted environment may be made.

Network system 800 is a network of devices (e.g., computers), each of which may be an example of data processing system 700, and other components. Network data processing system 800 may include network 802, which is a medium configured to provide communications links between various devices and computers connected within network data processing system 800. Network 802 may include connections such as wired or wireless communication links, fiber optic cables, and/or any other suitable medium for transmitting and/or communicating data between network devices, or any combination thereof.

In the depicted example, a first network device 804 and a second network device 806 connect to network 802, as do one or more computer-readable memories or storage devices 808. Network devices 804 and 806 are each examples of data processing system 700, described above. In the depicted example, devices 804 and 806 are shown as server computers, which are in communication with one or more server data store(s) 822 that may be employed to store information local to server computers 804 and 806, among others. However, network devices may include, without limitation, one or more personal computers, mobile computing devices such as personal digital assistants (PDAs), tablets, and smartphones, handheld gaming devices, wearable devices, tablet computers, routers, switches, voice gates, servers, electronic storage devices, imaging devices, media players, and/or other networked-enabled tools that may perform a mechanical or other function. These network devices may be interconnected through wired, wireless, optical, and other appropriate communication links.

In addition, client electronic devices 810 and 812 and/or a client smart device 814, may connect to network 802. Each of these devices is an example of data processing system 700, described above regarding FIG. 12. Client electronic devices 810, 812, and 814 may include, for example, one or more personal computers, network computers, and/or mobile computing devices such as personal digital assistants (PDAs), smart phones, handheld gaming devices, wearable devices, and/or tablet computers, and the like. In the depicted example, server 804 provides information, such as boot files, operating system images, and applications to one or more of client electronic devices 810, 812, and 814. Client electronic devices 810, 812, and 814 may be referred to as “clients” in the context of their relationship to a server such as server computer 804. Client devices may be in communication with one or more client data store(s) 820, which may be employed to store information local to the clients (e.g., cookie(s) and/or associated contextual information). Network data processing system 800 may include more or fewer servers and/or clients (or no servers or clients), as well as other devices not shown.

In some examples, first client electric device 810 may transfer an encoded file to server 804. Server 804 can store the file, decode the file, and/or transmit the file to second client electric device 812. In some examples, first client electric device 810 may transfer an uncompressed file to server 804 and server 804 may compress the file. In some examples, server 804 may encode text, audio, and/or video information, and transmit the information via network 802 to one or more clients.

Client smart device 814 may include any suitable portable electronic device capable of wireless communications and execution of software, such as a smartphone or a tablet. Generally speaking, the term “smartphone” may describe any suitable portable electronic device configured to perform functions of a computer, typically having a touchscreen interface, Internet access, and an operating system capable of running downloaded applications. In addition to making phone calls (e.g., over a cellular network), smartphones may be capable of sending and receiving emails, texts, and multimedia messages, accessing the Internet, and/or functioning as a web browser. Smart devices (e.g., smartphones) may include features of other known electronic devices, such as a media player, personal digital assistant, digital camera, video camera, and/or global positioning system. Smart devices (e.g., smartphones) may be capable of connecting with other smart devices, computers, or electronic devices wirelessly, such as through near field communications (NFC), BLUETOOTH®, WiFi, or mobile broadband networks. Wireless connectivity may be established among smart devices, smartphones, computers, and/or other devices to form a mobile network where information can be exchanged.

Data and program code located in system 800 may be stored in or on a computer-readable storage medium, such as network-connected storage device 808 and/or a persistent storage 708 of one of the network computers, as described above, and may be downloaded to a data processing system or other device for use. For example, program code may be stored on a computer-readable storage medium on server computer 804 and downloaded to client 810 over network 802, for use on client 810. In some examples, client data store 820 and server data store 822 reside on one or more storage devices 808 and/or 708.

Network data processing system 800 may be implemented as one or more of different types of networks. For example, system 800 may include an intranet, a local area network (LAN), a wide area network (WAN), or a personal area network (PAN). In some examples, network data processing system 800 includes the Internet, with network 802 representing a worldwide collection of networks and gateways that use the transmission control protocol/Internet protocol (TCP/IP) suite of protocols to communicate with one another. At the heart of the Internet is a backbone of high-speed data communication lines between major nodes or host computers. Thousands of commercial, governmental, educational and other computer systems may be utilized to route data and messages. In some examples, network 802 may be referred to as a “cloud.” In those examples, each server 804 may be referred to as a cloud computing node, and client electronic devices may be referred to as cloud consumers, or the like. FIG. 13 is intended as an example, and not as an architectural limitation for any illustrative embodiments.

### I. Illustrative Machine Learning Model

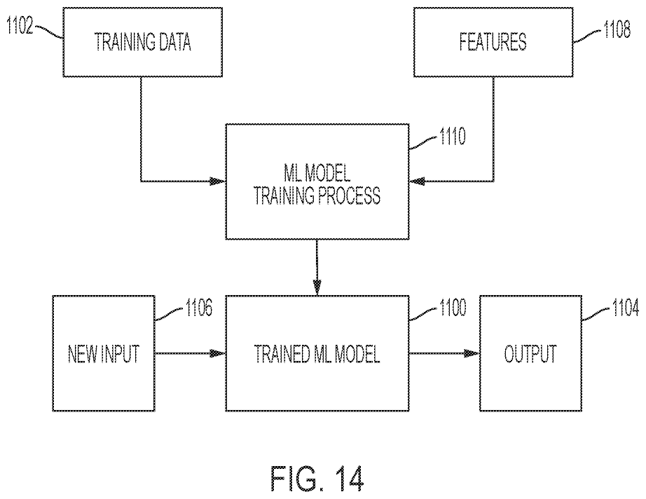

FIG. 14 depicts the training and use of an illustrative machine learning algorithm or model 1100. As mentioned above, machine learning algorithms may be utilized in one or more aspects of platform 100 (and/or other systems described herein).

In general, machine learning (ML) models (AKA ML algorithms, ML tools, or ML programs) may be utilized to generate predictions or decisions that are useful in themselves and/or in the service of a more comprehensive program. ML algorithms “learn” by example, based on existing sample data, and generate a trained model. Using the trained model, predictions or decisions can then be made regarding new data without explicit programming. Machine learning therefore involves algorithms or tools that learn from existing data and make predictions about novel data.

Training data 1102 (e.g., labeled training data) is utilized to build trained ML model 1100, such that the ML model can produce a desired output 1104 when presented with new data 1106. In general, the ML model uses labeled training data 1102, which includes values for the input variables and values for the known correct outputs, to ascertain relationships and correlations between variables or features 1108 to produce an algorithm mapping the input values to the outputs.

Supervised learning methods may be utilized for the purposes of producing classification or regression algorithms. Classification algorithms are typically used in situations where the goal is categorization (e.g., whether a photo contains a cat or a dog). Regression algorithms are typically used in situations where the goal is a numerical value (e.g., the market value of a house).

Features 1108 may include any suitable characteristics capable of being measured and configured to provide some level of information regarding the input scenario, situation, or phenomenon. For example, if the goal is to provide an output relating to the market value of a house, then the features may include variables such as square footage, postal code, year built, lot size, number of bedrooms, etc. Although these example features are numeric, other feature types may be included, such as strings, Boolean values, etc.

Different ML techniques may be used, depending on the application. For example, artificial neural networks, decision trees, support-vector machines, regression analysis, Bayesian networks, genetic algorithms, random forests, and/or the like may be utilized to produce the trained ML model.

Trained ML model 1100 is produced by training process 1110 based on identified features 1108 and training data 1102. Trained ML model 1100 can then be utilized to predict a category or decide an output value 1104 based on new data 1106.

With respect to the present disclosure, ML methods may be used in any suitable portion of platform 100. For example, a virtual coach may use ML methods to derive an insight about a user, to determine a response to present to a user in a chat session, and/or to accomplish any other suitable action. An analytics module of the platform may be configured to use ML methods to provide analytics and/or predictions about a user, about a group of users, and/or about any other suitable subject. Machine learning models of the platform may be trained using any suitable data, including, e.g., platform user data, platform knowledgebases, and/or any other suitable data described herein.

### J. Illustrative Combinations and Additional Examples

This section describes additional aspects and features of digital health platforms, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.

A0. A product comprising any feature described herein, either individually or in combination with any other such feature, in any configuration.

B0. A process for providing a user with a health or wellness service, the process comprising any process step described herein, in any order, using any modality.

C0. A computer-implemented health platform comprising:
a server including a server-side program configured to execute a virtual coach including an AI system;
a first client device including a client-side program in communication with the server-side program via a computer network;
wherein the client-side program and the server-side program are configured to facilitate a chat session between a user of the first client device and the virtual coach, and wherein facilitating the chat session includes:
   receiving, via a user interface executed at the first client device by the client-side program, a first user message input by the user;
   using the virtual coach, determining, based on first data including the first user message and user-specific data stored at a memory store in communication with the server, a first virtual coach message to be presented to the user;
   presenting, via the user interface, the first virtual coach message;
   receiving, via the user interface, a second user message input by the user;
   using the virtual coach, determining, based at least on the second user message, a second virtual coach message to be presented to the user; and
   presenting, via the user interface, the second virtual coach message.

C1. The platform of paragraph C0, wherein determining the first virtual coach message includes using a machine learning model, and wherein the first data is input to the machine learning model.

C2. The platform of paragraph C1, wherein determining the first virtual coach message includes deriving, using the machine learning model, a recommended action to be taken by the user; and wherein the first virtual coach message includes a recommendation to take the recommended action.

C3. The platform of any one of paragraphs C0-C2, further comprising using the virtual coach to identify a property of the user based on the first user message, wherein the first data used by the virtual coach to determine the first virtual coach message includes the identified property.

C4. The platform of paragraph C3, wherein the identified property of the user is a mood of the user, and identifying the property includes using a natural language processing model.

C5. The platform of any one of paragraphs C0-C4, wherein the server includes an analytics module configured to generate analytics data based on aggregated data associated with a plurality of users of the platform, and wherein the first data used by the virtual coach to determine the first virtual coach message includes the generated analytics data.

C6. The platform of paragraph C5, further comprising a second client device including an analytics program configured to facilitate access to the analytics module, the analytics program being configured to prevent access to the user-specific data.

C7. The platform of any one of paragraphs C0-C6, further comprising a third client device including a practitioner program, wherein the practitioner program and the server-side program are configured to facilitate a chat session between the user and a health practitioner.

C8. The platform of paragraph C7, wherein chat content input by the user and chat content input by the health practitioner are added to the user-specific data stored at the memory store, and wherein the first data used by the virtual coach to determine the first virtual coach message includes the stored chat content.

C9. The platform of any one of paragraphs C0-C8, wherein the server-side program further includes a course module including a plurality of courses each including health-related educational content, and wherein at least one of the first and second virtual coach messages comprises a recommendation to the user to access a first one of the courses.

C10. The platform of any one of paragraphs C0-C9, wherein the server-side program is configured to receive tracked user health data from a wearable electronic device of the user, and wherein the first data used by the virtual coach to determine the first virtual coach message includes the tracked user health data.

C11. The platform of any one of paragraphs C0-C10, wherein the server-side program is further configured to access information associated with an account of the user on a third-party social media service, and wherein the first data used by the virtual coach to determine the first virtual coach message includes data based on the accessed information.

C12. The platform of any one of paragraphs C0-C11, wherein the first data used by the virtual coach to determine the first virtual coach message includes metadata associated with one or more interactions between the user and the platform.

C13. The platform of paragraph C12, wherein the metadata includes at least one of: a frequency of user-initiated chat sessions with the virtual coach, a time of day when the user typically initiates chat sessions with the virtual coach, and a geographic location where the user typically initiates chat sessions with the virtual coach.

C14. The platform of any one of paragraphs C0-C13, wherein the user-specific data includes responses input by the user at the first client device to a plurality of questions relating to the user's physical health, the user's mental health, and at least one of: the user's spiritual health, the user's social health, the user's environmental health, and the user's economic health.

D0. A computer-implemented method for providing digital health and wellness services, the method comprising:
storing, at a memory store, user data relating to a wellness behavior of a user;
receiving, at a processor in communication with the memory store, a first chat message input by the user at a user computing device;
automatically deriving, based on the stored user data and the first chat message, using an artificial intelligence (AI) coach executed by the processor, a recommended action for the user to take to improve their wellness; and
presenting, at the user computing device, a second chat message including the recommended action.

D1. The method of paragraph D0, wherein the second chat message is generated by the AI coach using a machine learning model, wherein input to the machine learning model includes the first chat message and the stored user data.

D2. The method of paragraph D1, wherein the input to the machine learning model further includes aggregated data of a plurality of users.

D3. The method of any one of paragraphs D1-D2, wherein the AI coach is configured to identify a pattern relating to communication between the user computing device and the processor, and the input to the machine learning model further includes data representing the pattern.

D4. The method of any one of paragraphs D0-D3, wherein the AI coach is further configured to use natural language processing to automatically determine a mood of the user based on the first chat message, and wherein the second chat message is presented based in part on the determined mood.

### ADVANTAGES, FEATURES, AND BENEFITS

The different embodiments and examples of the system described herein provide several advantages over known solutions. For example, illustrative embodiments and examples described herein allow taking information from various sources and deriving AI based conclusions, recommendations etc.

The sources the information derives from include first-party data interaction possibilities.

Live data created from various sources can be used for obtaining quick studies.

Gained knowledge can be used to get an overall picture of certain user groups once enough users are on the platform.

Known digital health solutions in the market are highly fragmented. Health and wellness applications address only aspects of health—such as only exercise or only meditation or only mental health or only traditional medicine aspects of health, and so on. They are not treating the person as a whole and looking at a multitude of areas of one's health, and consequently, they are not integrating health disciplines. Likewise, these known solutions tend to treat people as one sizes fits all. Some variations are made for demographics—like age, height, weight—but they do not factor in lifestyle, health literacy, desire for change, health conditions, passions, interests, goals, constraints and limitations, etc. Likewise, AI solutions in this space are often symptom oriented and configured only to help someone diagnose their ailment. They help narrow down an illness or a disease. However, they do not identify the root causes of the disease or provide coaching or therapeutic guidance on living a healthy lifestyle and preventing the disease in the first place. Illustrative examples and embodiments disclosed herein address these problems by, e.g., providing services and assessments accounting for multiple dimensions of a person's health and wellbeing, and centralizing and integrating the knowledge, practices, and experiences of health disciplines from across the health spectrum to provide clients with whole-person solutions and health professionals with a knowledge base of integrative health best practices, research, information, and AI derived insights to optimize their care capabilities singly and collectively among the healthcare community worldwide.

An additional benefit of illustrative examples and embodiments of the present disclosure is that focusing on preventive solutions reduces the short- and long-term financial, physical, and psychological burdens of preventable illnesses on individuals, governments, and society at large. Compared to typical known health practitioner services, which are frequently expensive and include very small amounts of consultation time, for example 5-7 minutes, preventive health involves a deeper level of understanding of the individual's health status, life circumstances and involves root cause analysis. Likewise, preventive health involves educating the user and adopting the solutions that best matches their lifestyle, limitations, goals, and interests. Achieving these benefits takes longer than the 5-7 minutes typically available in conventional health care systems.

No known system or device can perform these functions. However, not all embodiments and examples described herein provide the same advantages or the same degree of advantage.

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

What is claimed is:

1. A computer-implemented health platform comprising:
a server including a server-side program configured to execute a virtual coach including an AI system;
a first client device including a client-side program in communication with the server-side program via a computer network;
wherein the client-side program and the server-side program are configured to facilitate a chat session between a user of the first client device and the virtual coach, and wherein facilitating the chat session includes:
receiving, via a user interface executed at the first client device by the client-side program, a first user message input by the user;
using the virtual coach, determining, based on first data including the first user message and user-specific data stored at a memory store in communication with the server, a first virtual coach message to be presented to the user;
presenting, via the user interface, the first virtual coach message;
receiving, via the user interface, a second user message input by the user;
using the virtual coach, determining, based at least on the second user message, a second virtual coach message to be presented to the user; and
presenting, via the user interface, the second virtual coach message.

2. The platform of claim 1, wherein determining the first virtual coach message includes using a machine learning model, and wherein the first data is input to the machine learning model.

3. The platform of claim 2, wherein determining the first virtual coach message includes deriving, using the machine learning model, a recommended action to be taken by the user; and wherein the first virtual coach message includes a recommendation to take the recommended action.

4. The platform of claim 1, further comprising using the virtual coach to identify a property of the user based on the first user message, wherein the first data used by the virtual coach to determine the first virtual coach message includes the identified property.

5. The platform of claim 4, wherein the identified property of the user is a mood of the user, and identifying the property includes using a natural language processing model.

6. The platform of claim 4, wherein the server includes an analytics module configured to generate analytics data based on aggregated data associated with a plurality of users of the platform, and wherein the first data used by the virtual coach to determine the first virtual coach message includes the generated analytics data.

7. The platform of claim 6, further comprising a second client device including an analytics program configured to facilitate access to the analytics module, the analytics program being configured to prevent access to the user-specific data.

8. The platform of claim 1, further comprising a third client device including a practitioner program, wherein the practitioner program and the server-side program are configured to facilitate a chat session between the user and a health practitioner.

9. The platform of claim 8, wherein chat content input by the user and chat content input by the health practitioner are added to the user-specific data stored at the memory store, and wherein the first data used by the virtual coach to determine the first virtual coach message includes the stored chat content.

10. The platform of claim 1, wherein the server-side program further includes a course module including a plurality of courses each including health-related educational content, and wherein at least one of the first virtual coach message and the second virtual coach message comprises a recommendation to the user to access a first one of the courses.

11. The platform of claim 1, wherein the server-side program is configured to receive tracked user health data from a wearable electronic device of the user, and wherein the first data used by the virtual coach to determine the first virtual coach message includes the tracked user health data.

12. The platform of claim 1, wherein the server-side program is further configured to access information associated with an account of the user on a third-party social media service, and wherein the first data used by the virtual coach to determine the first virtual coach message includes data based on the accessed information.

13. The platform of claim 1, wherein the first data used by the virtual coach to determine the first virtual coach message includes metadata associated with one or more interactions between the user and the platform.

14. The platform of claim 13, wherein the metadata includes at least one of: a frequency of user-initiated chat sessions with the virtual coach, a time of day when the user typically initiates chat sessions with the virtual coach, and a geographic location where the user typically initiates chat sessions with the virtual coach.

15. The platform of claim 1, wherein the user-specific data includes responses input by the user at the first client device to a plurality of questions relating to the user's physical health, the user's mental health, and at least one of: the user's spiritual health, the user's social health, the user's environmental health, and the user's economic health.

16. A computer-implemented method for providing digital health and wellness services, the method comprising:
storing, at a memory store, user data relating to a wellness behavior of a user;
receiving, at a processor in communication with the memory store, a first chat message input by the user at a user computing device;
automatically deriving, based on the stored user data and the first chat message, using an artificial intelligence (AI) coach executed by the processor, a recommended action for the user to take to improve their wellness; and
presenting, at the user computing device, a second chat message including the recommended action.

17. The method of claim 16, wherein the second chat message is generated by the AI coach using a machine learning model, wherein input to the machine learning model includes the first chat message and the stored user data.

18. The method of claim 17, wherein the input to the machine learning model further includes aggregated data of a plurality of users.

19. The method of claim 17, wherein the AI coach is configured to identify a pattern relating to communication between the user computing device and the processor, and the input to the machine learning model further includes data representing the pattern.

20. The method of claim 16, wherein the AI coach is configured to use natural language processing to automatically determine a mood of the user based on the first chat message, and wherein the second chat message is presented based in part on the determined mood.
